(19) United States
(12) Patent Application Publication
(10) Pub. No.: US 2005/0095224 A1
(43) Pub. Date: May 5, 2005

(54) Compositions and method for treating hepatitis virus infection

(76) Inventors: Ramachandran Radhakrishnan, Fremont, CA (US); Gary Visor, San Diego, CA (US); Peter Van Vlasselaer, Portola Valley, CA (US); Richard B. Love, Montara, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/496,999

(22) PCT Filed: Dec. 5, 2002
(86) PCT No.: PCT/US02/39101

Related U.S. Application Data
(60) Provisional application No. 60/338,991, filed on Dec. 7, 2001.

(51) Int. Cl. A61K 38/21

(52) U.S. Cl. 424/85.7; 530/351

(57) ABSTRACT

The present invention provides compositions and methods of treating hepatitis virus infection, particularly hepatitis C virus infection. The invention provides methods of treating a hepatitis virus infection, involving administering a first form and a second form of IFN-α to provide a multiphasic pharmacokinetic profile. The multiphasic antiviral agent serum concentration profile that is achieved effects an initial rapid drop in viral titer, followed by a further decrease in viral titer over time, to achieve a sustained viral response. The invention further provides compositions that are effective in achieving a multiphasic IFN-α profile. Compositions of the invention comprise at least a first form of interferon-α (IFN-α) that has a first pharmacokinetic profile and a second form of IFN-α that has a second pharmacokinetic profile, where the second form of IFN-α has a longer mean residence time than that of the first form of IFN-α. The invention further provides compositions comprising C-terminally modified IFN-α.

### FIELD OF THE INVENTION

This invention is in the field of treatments for viral infections, in particular hepatitis virus.

### BACKGROUND OF THE INVENTION

Hepatitis C virus (HCV) infection is the most common chronic blood borne infection in the United States. Although the numbers of new infections have declined, the burden of chronic infection is substantial, with Centers for Disease Control estimates of 3.9 million (1.8%) infected persons in the United States. Chronic liver disease is the tenth leading cause of death among adults in the United States, and accounts for approximately 25,000 deaths annually, or approximately 1% of all deaths. Studies indicate that 40% of chronic liver disease is HCV-related, resulting in an estimated 8,000-10,000 deaths each year. HCV-associated end-stage liver disease is the most frequent indication for liver transplantation among adults.

Antiviral therapy of chronic hepatitis C has evolved rapidly over the last decade, with significant improvements seen in the efficacy of treatment. Nevertheless, even with combination therapy using PEGylated IFN-α plus ribavirin, 40% to 50% of patients fail therapy, i.e., are nonresponders or relapsers. These patients currently have no effective therapeutic alternative. In particular, patients who have advanced fibrosis or cirrhosis on liver biopsy are at significant risk of developing complications of advanced liver disease, including ascites, jaundice, variceal bleeding, encephalopathy, and progressive liver failure, as well as a markedly increased risk of hepatocellular carcinoma.

The high prevalence of chronic HCV infection has important public health implications for the future burden of chronic liver disease in the United States. Data derived from the National Health and Nutrition Examination Survey (NHANES III) indicate that a large increase in the rate of new HCV infections occurred from the late 1960s to the early 1980s, particularly among persons between 20 to 40 years of age. It is estimated that the number of persons with long-standing HCV infection of 20 years or longer could more than quadruple from 1990 to 2015, from 750,000 to over 3 million. The proportional increase in persons infected for 30 or 40 years would be even greater. Since the risk of HCV-related chronic liver disease is related to the duration of infection, with the risk of cirrhosis progressively increasing for persons infected for longer than 20 years, this will result in a substantial increase in cirrhosis-related morbidity and mortality among patients infected between the years of 1965-1985.

Chronic hepatitis C virus infection is characterized by intermittent or persistent elevations in serum alanine aminotransferase (ALT) levels and constant levels of HCV RNA in the circulation. Currently, approved therapies use alpha interferons derived from natural leukocytes or by recombinant methods using cDNA sequences of specific subtypes or consensus interferon-α (IFN-α). The accepted dosage regimen is a subcutaneous administration of IFN-α in the dose ranges of 6-50 μg three times in week for a period of 24-48 weeks.

Cyclical administration of IFN-α has also been conducted, in the hope that viral clearance can be achieved. The repeat dosing has been deemed necessary in view of the rapid clearance and in vivo degradation of IFN-α. In another attempt to achieve better efficacy, combination therapies such as IFN-α and ribavirin have been carried out. Recent interim results from a Phase IV clinical trial comparing the use of Infergen plus ribavirin to the use of interferon alfa-2b plus ribavirin (Rebetron™) indicate that some of the interferons may be more potent in achieving a sustained viral response (SVR) than others. For example, patients treated with Infergen in combination with ribavirin achieved and SVR of 56% compared with an SVR of 31% in patients treated with Rebetron.

In attempts to improve further the therapeutic methods, various investigators have attempted a chemical modification of IFN-α by adding a polymer chain(s) to increase the molecular weight and size of the protein and to prolong the systemic circulation times. While these manipulations of IFN-α increased the circulation times and improved the efficacies further, a significant fraction of the protein loses its biological activity. Thus higher amounts of the protein have to be delivered to the patient with adverse effects such as neutropenia accompanying such administrations.

An example of such modification of IFN-α is addition of polyethylene glycol (PEG) chains to the IFN-α molecule, in a process known as “PEGylation.” The PEGylation of alpha interferons can lead to a significant reduction in the antiviral activity of the polypeptide, and thus PEGylation must be carefully controlled to avoid modification of residues that may result in an undesirable reduction of activity. For example, chemical modifications in the receptor binding domains in the interferon molecule, such as the AB loop (residues 29-35), helix D (123-140) and subtype differentiating domain (residues 78-95), lead to significant losses in the antiviral activity of the protein. Mutagenesis, deletion, chemical modification and nuclear magnetic resonance studies have shown that the lysines or histidines in these domains such as His 34 are critical determinants for activity.

Viral kinetics during treatment regimens that include IFN-α have been examined. In general, an initial rapid decline in viral titers (early viral response; EVR) is seen in some individuals. The EVR results in an approximately 0.5- to 3-log decrease in serum HCV RNA levels in a period of 24-48 hours after initiation of treatment. An early robust response is favorable toward achieving a durable response. In some individuals, the EVR is followed by a further, less rapid decline of the virus in blood (second phase decline). The second phase decline is a slower decrease in the level of the virus over several weeks or months.

Despite the availability of approved treatment regimens discussed above, only a small fraction of the individuals treated attain a sustained viral response. Thus, there is a need in the art for improved methods for treating HCV infection. The present invention addresses this need.

Literature

U.S. Pat. Nos. 6,172,046; 6,245,740; 5,824,784; 5,372,808; 5,980,884; published international patent applications WO 96/21468; WO 96/11953; Torre et al. (2001) J. Med. Virol. 64:455-459; Bekkering et al. (2001) J. Hepatol. 34:435-440; Zeuzem et al. (2001) Gastroenterol. 120:1438-1447; Zeuzem (1999) J. Hepatol. 31:61-64; Keeffe and Hollinger (1997) Hepatol. 26:101S-107S; Wills (1990) Clin. Pharmacokinet. 19:390-399; Heathcote et al. (2000) New Engl. J. Med. 343:1673-1680; Husa and Husova (2001) Bratisl. Lek. Listy 102:248-252; Glue et al. (2000) Clin. Pharmacol. 68:556-567; Bailon et al. (2001) Bioconj. Chem. 12:195-202; and Neumann et al. (2001) Science 282:103; Zalipsky (1995) Adv. Drug Delivery Reviews S. 16, 157-182; Mann et al. (2001) Lancet 358:958-965; Zeuzem et al. (2000) New Engl. J. Med 343:1666-1672; U.S. Pat. Nos. 5,985,265; 5,908,121; 6,177,074; 5,985,263; 5,711,944; 5,382,657; and 5,908,121.

### SUMMARY OF THE INVENTION

The present invention provides compositions and methods of treating hepatitis virus infection, particularly hepatitis C virus infection. The invention provides methods of treating a hepatitis virus infection, involving administering a first form and a second form of IFN-α to provide a multiphasic pharmacokinetic profile. The multiphasic antiviral agent serum concentration profile that is achieved effects an initial rapid drop in viral titer, followed by a further decrease in viral titer over time, to achieve a sustained viral response. The invention further provides compositions that are effective in achieving a multiphasic IFN-α profile. Compositions of the invention comprise at least a first form of interferon-α (IFN-α) that has a first pharmacokinetic profile and a second form of IFN-α that has a second pharmacokinetic profile, where the second form of IFN-α has a longer mean residence time than that of the first form of IFN-α. The invention further provides compositions comprising C-terminally modified IFN-α.

### Features of the Invention

Treatment Methods

The present invention features a method for treating hepatitis C virus infection in an individual. The method generally involves administering a composition comprising a first form of interferon-α (IFN-α) and a second form of IFN-α, wherein said second form of IFN-α comprises a polyethylene glycol (PEG) moiety and, as a result, has a mean residence time that is greater than the mean residence time of the first form of IFN-α, which composition is administered in an amount effective to achieve a first serum concentration of IFN-α that is at least about 80% of the maximum tolerated dose (MTD) in International Units of IFN-α per milliliter of serum (IU/ml) within a first period of time of about 24 to 48 hours, followed by a second concentration of IFN-α that is about 50% or less than the MTD, which second concentration is maintained for a second period of time of at least seven days. In many embodiments, a sustained viral response is achieved.

In some embodiments, the method further involves administering IFN-γ for a period of from about 1 day to about 14 days before administration of IFN-α.

In some embodiments, the second form of IFN-α comprises a PEG moiety covalently linked, directly or via a linker, to one or more amino acid side chains of amino acid residues 1-10 of the IFN-α polypeptide.

In some embodiments, the second form of IFN-α comprises a PEG moiety covalently linked, directly or via a linker, to the amino-terminal amino acid of the IFN-α polypeptide. In some embodiments, the second form of IFN-α comprises a PEG moiety covalently linked, directly or via a linker, to one or more amino acid side chains of amino acid residues 150-166 of the IFN-α polypeptide. In some embodiments, the second form of IFN-α comprises a PEG moiety covalently linked, directly or via a linker, to the carboxyl-terminal amino acid of the IFN-α polypeptide.

The invention further features a method of treating hepatitis C virus infection in an individual. The method generally involves administering a composition comprising a first form of interferon-α (IFN-α) and a second form of IFN-α, wherein said second form of IFN-α comprises a polyethylene glycol (PEG) moiety and, as a result, has a mean residence time that is greater than the mean residence time of the first form of IFN-α, wherein the composition is administered in an amount effective to achieve a first phase and a second phase, wherein, in the first phase, a first serum concentration of IFN-α is achieved that is at least about 80% of the maximum tolerated dose (MTD) in International Units of IFN-α per milliliter of serum (IU/ml) within a first period of time of about 24 hours, wherein in the second phase, the ratio of the highest IFN-α serum concentration to the lowest serum IFN-α concentration, measured over any 24-hour period during the second phase, is less than 3, and wherein the highest concentration of IFN-α during the second phase is about 50% or less than the MTD.

In some embodiments, the ratio of the highest IFN-α serum concentration to the lowest serum IFN-α concentration, measured over any 24-hour period during the second phase is about 1.

The invention further features a method for treating hepatitis C virus infection in an individual. The method generally involves administering a composition comprising a first form of consensus interferon-α (CIFN) and a second form of CIFN, wherein said second form of CIFN comprises a polyethylene glycol (PEG) moiety and, as a result, has a mean residence time that is greater than the mean residence time of the first form of CIFN, wherein the composition is administered in an amount effective to achieve a first serum concentration of CIFN that is at least about 80% of the maximum tolerated dose (MTD) in International Units of IFN-α per milliliter of serum (IU/ml) within a first period of time of about 24 hours, followed by a second concentration of CIFN that is about 50% or less than the MTD, which second concentration is maintained for a second period of time of at least seven days.

The invention further features a method of treating hepatitis C virus infection in an individual. The method generally involves administering a composition comprising a first form of consensus interferon-α (CIFN) and a second form of CIFN, wherein said second form of CIFN comprises a polyethylene glycol (PEG) moiety and, as a result, has a mean residence time that is greater than the mean residence time of the first form of CIFN, wherein the composition is administered in an amount effective to achieve a first phase and a second phase, wherein, in the first phase, a first serum concentration of CIFN is achieved that is at least about 80% of the maximum tolerated dose (MTD) in International Units of IFN-α per milliliter of serum (IU/ml) within a first period of time of about 24 hours, wherein in the second phase, the ratio of the highest CIFN serum concentration to the lowest serum CIFN concentration, measured over any 24-hour period during the second phase, is less than 3, and wherein the highest concentration of CIFN during the second phase is about 50% or less than the MTD.

The invention further features a method of treating hepatitis C virus infection in an individual. The method generally involves administering IFN-α in a dosing regimen comprising a first phase and a second phase, wherein, in the first phase, a first serum concentration C Imax of IFN-α in International Units of IFN-α per milliliter of serum (IU/ml) is achieved within a first period of time of about 24 hours, wherein in the second phase, a second serum concentration Csus in International Units of IFN-α per milliliter of serum (IU/ml) is achieved that is about 50% of C1max or less, and wherein the area under the curve, defined by IFN-α serum concentration as a function of time, during any 24-hour time period in the second phase is no greater than the area under the curve of day 2 to day 3 as shown in FIG. 2.

The invention further features a method of treating hepatitis C virus infection in an individual. The method generally involves administering consensus IFN-α (CIFN) in a dosing regimen comprising a first phase and a second phase, wherein, in the first phase, a first serum concentration C1max of CIFN in International Units of IFN-α per milliliter of serum (IU/ml) is achieved within a first period of time of about 24 hours, wherein in the second phase, a second concentration Csus of CIFN in International Units of IFN-α per milliliter of serum (IU/ml) is achieved that is about 50% of C Imax or less, and wherein the area under the curve, defined by CIFN serum concentration as a function of time, during any 24-hour time period in the second phase is no greater than the area under the curve of day 2 to day 3 as shown in FIG. 2.

The invention further features a method of treating hepatitis C infection in an individual. The method generally involves administering an effective amount of a subject composition to the individual. In some embodiments, the subject composition comprises: a first form of interferon-α (IFN-α), wherein the covalent molecular structure of the first form of IFN-α comprises a first IFN-α polypeptide free of polyethylene glycol; a second form of IFN-α, wherein the covalent molecular structure of the second form of IFN-α comprises a second IFN-α polypeptide covalently linked, directly or via a linker, to a polyethylene glycol (PEG) moiety; and a pharmaceutically acceptable excipient. In other embodiments, the subject composition comprises an interferon-α (IFN-α) derivative comprising a single interferon-α (IFN-α) polypeptide, wherein the IFN-α polypeptide is covalently linked, directly or via a linker, to one or more polyethylene glycol (PEG) moieties, wherein the IFN-α polypeptide is linked to each PEG moiety at one or more sites at or near the carboxyl-terminus of the IFN-α polypeptide, and wherein the IFN-α polypeptide is covalently linked, directly or via a linker, to no PEG moiety at any site other than a site at or near the carboxyl-terminus of the IFN-α polypeptide. In some embodiments, the administration of the subject composition delivers to the individual a total of from about 0.5×10⁶ to 10×10⁶ IU IFN-α. In some embodiments, the administration of the subject composition delivers to the individual a total of about 5,000,000 to 10,000,000 International Units of interferon-α.

Compositions Comprising a First and a Second Form of IFN-α

The invention further features a composition comprising: a first form of interferon-α (IFN-α), wherein the covalent molecular structure of the first form of IFN-α comprises a first IFN-α polypeptide free of polyethylene glycol; a second form of IFN-α, wherein the covalent molecular structure of the second form of IFN-α comprises a second IFN-α polypeptide covalently linked, directly or via a linker, to a polyethylene glycol (PEG) moiety; and a pharmaceutically acceptable excipient.

In some embodiments, in the second form of IFN-α, the PEG moiety is covalently linked, directly or via a linker, to one or more amino acid side chains of amino acid residues 1-10 of the second IFN-α polypeptide. In some embodiments, in the second form of IFN-α, the PEG moiety is covalently linked, directly or via a linker, to the amino-terminal amino acid of the second IFN-α polypeptide.

In some embodiments, the PEG moiety is covalently linked, directly or via a linker, to the α-amino group of the amino-terminal amino acid of the second polypeptide. In some embodiments, the PEG moiety is covalently linked, directly or via a linker, by an amide bond to the α-amino group of the amino-terminal amino acid of the second polypeptide.

In some embodiments, in the second form of IFN-α, the PEG moiety is covalently linked, directly or via a linker, to one or more amino acid side chains of amino acid residues 150-166 of the second IFN-α polypeptide.

In some embodiments, in the second form of IFN-α, the PEG moiety is covalently linked, directly or via a linker, to the carboxyl-terminal amino acid of the second IFN-α polypeptide. In some embodiments, the PEG moiety is covalently linked, directly or via a linker, to the α-carboxyl group of the carboxyl-terminal amino acid of the second IFN-α polypeptide. In some embodiments, the PEG moiety is covalently linked, directly or via a linker, by an amide bond to the α-carboxyl group of the carboxyl-terminal amino acid of the second IFN-α polypeptide.

In some embodiments, the covalent molecular structure of the second form of IFN-α comprises no PEG moiety that is linked, directly or via a linker, to an amino acid in amino acid residues 1-149 of the second IFN-α polypeptide.

In some embodiments, the second form of IFN-α comprises a single PEG moiety.

In some embodiments, the first form of IFN-α and the second form of IFN-α are present at a molar ratio of about 1:1 in the composition. In some embodiments, the first form of IFN-α and the second form of IFN-α are present at a molar ratio of about 1:5 in the composition.

In some embodiments, the first form of IFN-α and the second form of IFN-α each comprise a single IFN-α polypeptide molecule, wherein the IFN-α polypeptide is the same for the first and second forms and is selected from the group consisting of IFN-α-2a, IFN-α-2b and consensus IFN-α polypeptides. In some embodiments, the IFN-α polypeptide is selected from the group consisting of consensus IFN-α polypeptides.

In some embodiments, the first form of IFN-α and the second form of IFN-α each comprise a single IFN-α polypeptide molecule, wherein the IFN-α polypeptide is the same for the first and second forms and is selected from the group consisting of consensus IFN-α polypeptides.

In some embodiments, the second form of IFN-α comprising a single PEG moiety.

IFN-α Derivatives

The invention further features an interferon-α (IFN-α) derivative comprising a single interferon-α (IFN-α) polypeptide, wherein the IFN-α polypeptide is covalently linked, directly or via a linker, to one or more polyethylene glycol (PEG) moieties, wherein the IFN-α polypeptide is linked to each PEG moiety at one or more sites at or near the carboxyl-terminus of the IFN-α polypeptide, and wherein the IFN-α polypeptide is covalently linked, directly or via a linker, to no PEG moiety at any site other than a site at or near the carboxyl-terminus of the IFN-α polypeptide.

The invention further features an interferon-α (IFN-α) derivative comprising a single interferon-α (IFN-α) polypeptide, wherein the IFN-α polypeptide is either (1) covalently linked to a single PEG moiety directly, or via a linker, via a single covalent bond located at or near the carboxyl-terminus of the IFN-α polypeptide or (2) covalently linked to a plurality of PEG moieties via a linker and via a single covalent bond between the linker and the IFN-α polypeptide, wherein the bond is located at a site at or near the carboxyl-terminus of the IFN-α polypeptide.

In some embodiments, the IFN-α polypeptide is covalently linked, directly or via a linker, to no amino acid in amino acid residues 1-149 of the IFN-α polypeptide.

In some embodiments, at least one PEG moiety is covalently linked, directly or via a linker, to one or more amino acid side chains of amino acid residues 150-166 of the IFN-α polypeptide.

In some embodiments, at least one PEG moiety is covalently linked, directly or via a linker, to the carboxyl-terminal amino acid of the IFN-α polypeptide. In some embodiments, at least one PEG moiety is covalently linked, directly or via a linker, to the α-carboxyl group of the carboxyl-terminal amino acid of the IFN-α polypeptide. In some embodiments, at least one PEG moiety is covalently linked, directly or via a linker, by an amide bond to the α-carboxyl group of the carboxyl-terminal amino acid of the IFN-α polypeptide.

In some embodiments, the IFN-α polypeptide is covalently linked to a single PEG moiety.

In some embodiments, the IFN-α polypeptide is a consensus interferon-α polypeptide. In some embodiments, the IFN-α polypeptide is an interferon-α-2a polypeptide. In some embodiments, the IFN-α polypeptide is an interferon-α-2b polypeptide.

The invention further features a composition comprising a subject interferon-α (IFN-α) derivative; and a pharmaceutically acceptable excipient. In some embodiments, the IFN-α derivative comprises a single PEG moiety covalently linked, directly or through a linker, at or near the carboxyl terminus of the IFN-α polypeptide; and a pharmaceutically acceptable excipient. In some embodiments, the IFN-α polypeptide is a consensus interferon-α polypeptide.

### BRIEF DESCRIPTION OF THE DRAWINGS

FIG. 1 is a graph depicting viral kinetics during interferon-α therapy depicted here as clearance of HCV virus in blood as monitored by the level of viral RNA in serum using a sensitive measurement such as a polymerase chain reaction.

FIG. 2 is a graph depicting a profile of serum IFN-α concentration during administration of a controlled Release Injectible (CR1) system or a zero-order throughput system and bolus. Viral kinetics following conventional TIW regimen is included.

FIG. 3 is a graph depicting an exemplary pharmacokinetic profile of serum IFN-α concentration after administration of a first form of IFN-α and a second form of IFN-α according to the invention.

FIG. 4 is a graph depicting an exemplary pharmacokinetic profile of serum IFN-α concentration after administration of a first form of IFN-α and a second form of IFN-α according to the invention.

FIG. 5 is a graph depicting an exemplary pharmacokinetic profile of serum IFN-α concentration after administration of a first form of IFN-α and a second form of IFN-α according to the invention.

### DEFINITIONS

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease (as in liver fibrosis that can result in the context of chronic HCV infection); (b) inhibiting the disease, i.e., arresting its development; and (c) relieving the disease, i.e., causing regression of the disease.

The terms “individual,” “host,” “subject,” and “patient” are used interchangeably herein, and refer to a mammal, including, but not limited to, primates, including simians and humans.

The term “early viral response (EVR),” used interchangeably with “initial viral response,” “rapid viral response” refers to the drop in viral titer within about 24 hours, about 48 hours, about 3 days, or about 1 week after the beginning of treatment for HCV infection.

The term “second phase decline” as used herein refers to a slower decrease in the level of the virus over several weeks or months after the EVR.

The term “sustained viral response” (SVR; also referred to as a “sustained response” or a “durable response”), as used herein, refers to the response of an individual to a treatment regimen for HCV infection, in terms of serum HCV titer. Generally, a “sustained viral response” refers to no detectable HCV RNA (e.g., less than about 500, less than about 200, or less than about 100 genome copies per milliliter serum) found in the patient's serum for a period of at least about one month, at least about two months, at least about three months, at least about four months, at least about five months, or at least about six months following cessation of treatment.

The term “pharmacokinetic profile,” as used herein, refers to the profile of the serum concentration of IFN-α over time.

“Treatment failure patients” as used herein generally refers to HCV-infected patients who failed to respond to previous therapy for HCV (referred to as “non-responders”) or who initially responded to previous therapy, but in whom the therapeutic response was not maintained (referred to as “relapsers”). The previous therapy generally can include treatment with IFN-α monotherapy or IFN-α combination therapy, where the combination therapy may include administration of IFN-α and an antiviral agent such as ribavirin.

The term “hepatitis virus infection” refers to infection with one or more of hepatitis A, B, C, D, or E virus, with blood-borne hepatitis viral infection being of particular interest.

As used herein, the term “hepatic fibrosis,” used interchangeably herein with “liver fibrosis,” refers to the growth of scar tissue in the liver that can occur in the context of a chronic hepatitis infection.

As used herein, the term “liver function” refers to a normal function of the liver, including, but not limited to, a synthetic function, including, but not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

Drug delivery devices that are suitable for use in the subject methods include, but are not limited to, injection devices; an implantable device, e.g., pumps, such as an osmotic pump, that may or may not be connected to a catheter; biodegradable implants; liposomes; depots; and microspheres.

The term “dosing event” as used herein refers to administration of an antiviral agent to a patient in need thereof, which event may encompass one or more releases of an antiviral agent from a drug dispensing device. Thus, the term “dosing event,” as used herein, includes, but is not limited to, installation of a depot comprising an antiviral agent; installation of a continuous delivery device (e.g., a pump or other controlled release injectible system); and a single subcutaneous injection followed by installation of a continuous delivery system.

The term “depot” refers to any of a number of implantable, biodegradable or non-biodegradable, controlled release systems that are generally non-containerized and that act as a reservoir for a drug, and from which drug is released. Depots include polymeric non-polymeric biodegradable materials, and may be solid, semi-solid, or liquid in form.

The term “microsphere” (also referred to as “microparticles,” “nanospheres,” or “nanoparticles”) refers to small particles, generally prepared from a polymeric material and usually having a size in the range of from about 0.01 μm to about 0.1 μm, or from about 0.1 μm to about 10 μm in diameter.

The term “therapeutically effective amount” is meant an amount of a therapeutic agent, or a rate of delivery of a therapeutic agent, effective to facilitate a desired therapeutic effect. The precise desired therapeutic effect will vary according to the condition to be treated, the formulation to be administered, and a variety of other factors that are appreciated by those of ordinary skill in the art.

The terms “International Units” and “Units” are used interchangeably herein to refer to units of measurement for quantitation of the ability of the interferon to inhibit the cytopathic effect of a suitable virus (e.g. encephalomyocarditis virus (EMC), vesicular stomatitis virus, Semliki forest virus) after infection of an appropriate cell line (e.g., the human lung carcima cell lines, A549; HEP2/C; and the like). The antiviral activity is normalized to “Units” of antiviral activity exhibited by a reference standard such as human interferon alpha supplied by WHO. Such methods are detailed in numerous references. A particular method for measuring International Units is described in Familletti, P. C., Rubinstein, S and Pestka, S.(1981) “A convenient and rapid cytopathic effect inhibition assay for interferon”, Methods in Enzymol, Vol 78 (S. Pestka, ed), Academic Press, New York pages 387-394. For the most part, the reference standard is human interferon alpha supplied by the World Health Organization, and the method for measuring International Units is that described in Familletti, supra.

The amounts of interferon administered will depend on the specific activities of the compounds and their biological performance in vivo. For example, IFN-α 2b is administered at 11.54 μg protein three times a week corresponding to 3×10⁶ IU per injection (specific activity, 2.68×10⁶ IU/mg). On the other hand, CIFN alfa-con 1 is administered at 9 μg doses per injection corresponding to 9×10⁶ IU per administration (specific activity, 1×10⁹ IU/mg). However, in view of the fact that PEGylation reactions often result in a reduction in activity, larger mass doses of PEGylated material are administered to achieve efficacy (e.g. reduction in viral load; sustained viral response, etc.).

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “an interferon-alpha polypeptide” includes a plurality of such polypeptides and reference to “the pharmacokinetc profile” includes reference to one or more pharmacokinetic profiles and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention provides compositions and methods of treating hepatitis virus infection, particularly hepatitis C virus (HCV) infection. The methods involve administering a first form of interferon-α (IFN-α) that has a first pharmacokinetic profile and at least a second form of IFN-α that has a second pharmacokinetic profile. The second form of IFN-α has a longer mean residence time (or serum half-life) that that of the first form of IFN-α. Thus, the combined pharmacokinetic profiles of the first and the second forms of IFN-α achieve a multiphasic serum concentration profile of IFN-α. The multiphasic IFN-α serum concentration profile that is achieved effects an initial rapid drop in viral titer, followed by a further decrease in viral titer over time, to achieve a sustained viral response. The multiphasic serum concentration profile are achieved by administering the first and second forms of IFN-α substantially simultaneously but in separate formulations; simultaneously in the same formulation; or at separate time points in separate formulations.

The methods and compositions described herein are generally useful in treatment of any hepatitis viral infection (HBV, HCV, delta, etc.). Treatment of HCV infection is of particular interest. Reference to HCV herein is for illustration only and is not meant to be limiting.

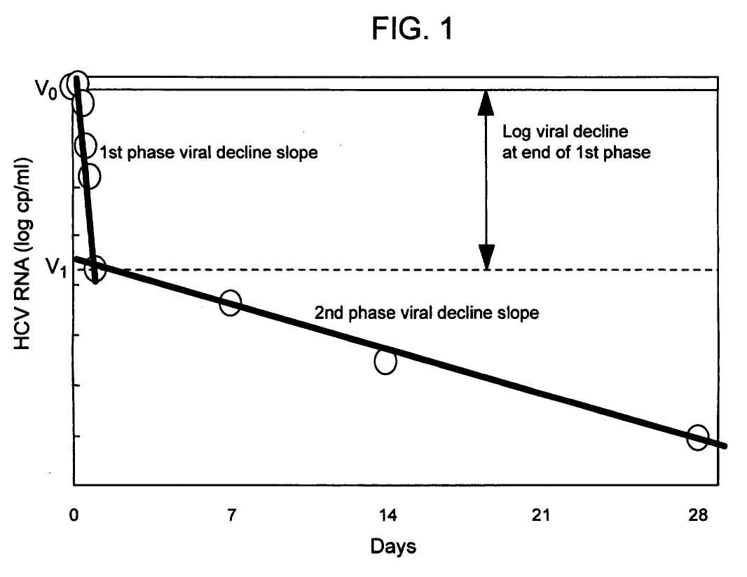

Currently available IFN-α therapies for treating HCV infection generally involve subcutaneous injections of IFN-α daily (QD), every other day (QOD), or three times a week (TIW). The kinetics of HCV infection among responders in response to conventional IFN-α therapies, as determined by RNA PCR, have been analyzed by mathematical modeling. The general interpretation of such results is represented in FIG. 1. Such studies have clearly shown a rapid viral decline phase in 24-48 hours after the beginning of treatment, resulting in an approximately 0.5-log to an approximately 3-log or greater decrease in serum RNA levels. This early viral response (EVR) is important in reducing the production of viral particles. An early, robust response is generally predictive of a more durable response. This early phase is usually followed by a slower, sustained clearance of the virus over several days or weeks. Generally, this second phase is dependent on characteristics associated with the patient. Without wishing to be bound by any one theory, the second phase reduction in viral titer may be related to removal of virus-infected cells, e.g., by immune system mediated mechanisms. The slope of this second phase is determinative of the sustained viral response (SVR) of the patient, e.g., a steeper second phase slope is generally associated with a SVR and a positive treatment outcome.

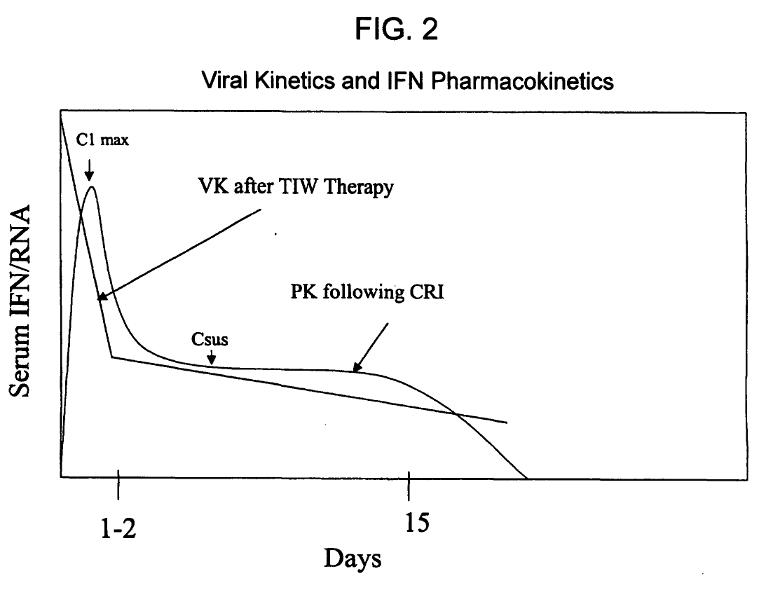

Current therapies to treat HCV infection suffer from certain drawbacks. Dosing regimens involving daily (QD), every other day (QOD), or thrice weekly (TIW) injections of IFN-α over extended treatment periods suffer from one or more of the following drawbacks: (1) the dosing regimens are uncomfortable to the patient and, in some cases, result in reduced patient compliance; (2) the dosing regimens are often associated with adverse effects, causing additional discomfort to the patient, and, in some cases, resulting in reduced patient compliance; (3) the dosing regimens result in “peaks” (Cmax) and “troughs” (Cmin) in serum IFN-α concentration, and, during the “trough” periods, virus can replicate, and/or infect additional cells, and/or mutate; (4) in many cases, the log reduction in viral titer during the early viral response is insufficient to effect a sustained viral response that ultimately results in clearance of the virus (see FIG. 2; viral kinetics after conventional IFN-α TIW therapy).

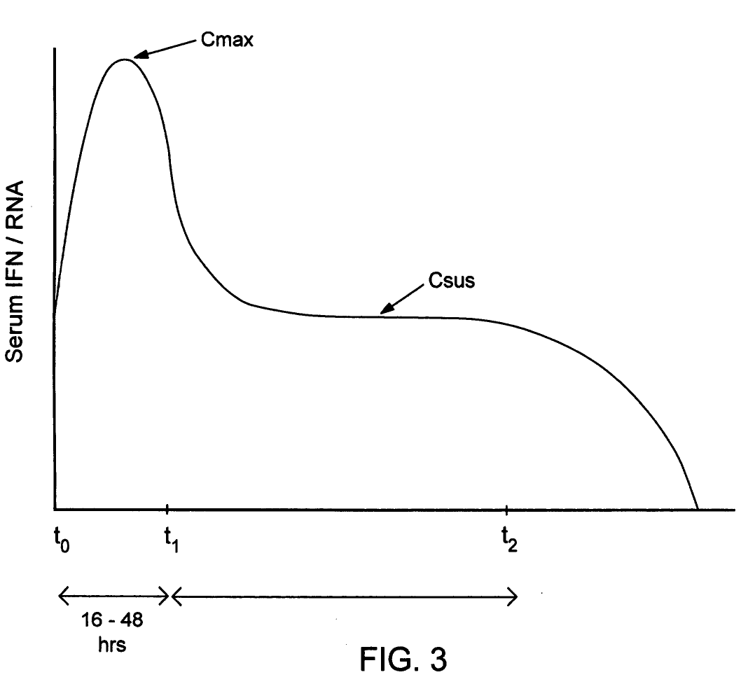

The methods of the instant invention involve administering a first form of IFN-α and a second form of IFN-α to achieve a multiphasic serum IFN-α that results in a sustained viral response. An exemplary pharmacokinetic profile to achieved through the present invention is illustrated in FIG. 3, where Cmax is the concentration of IFN-α that is achieved in an “induction phase” and Csus is the concentration of IFN-α that is achieve in a “maintenance phase.”

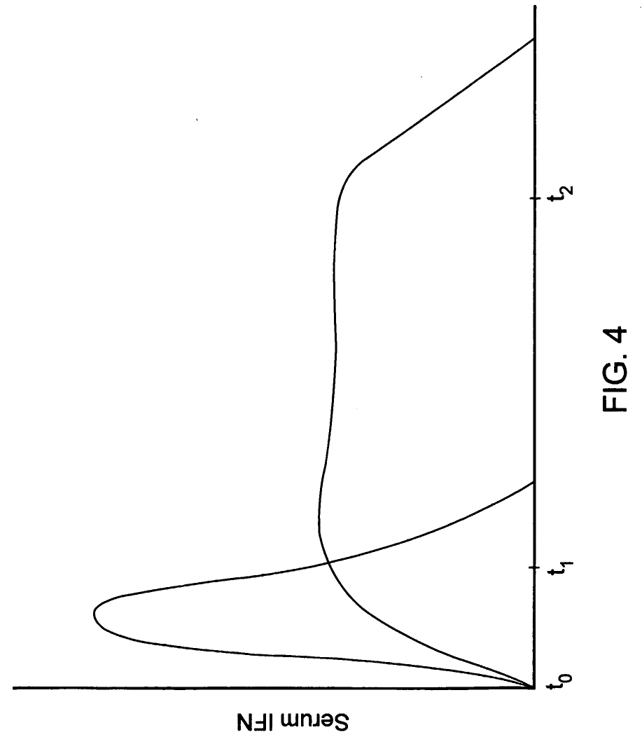

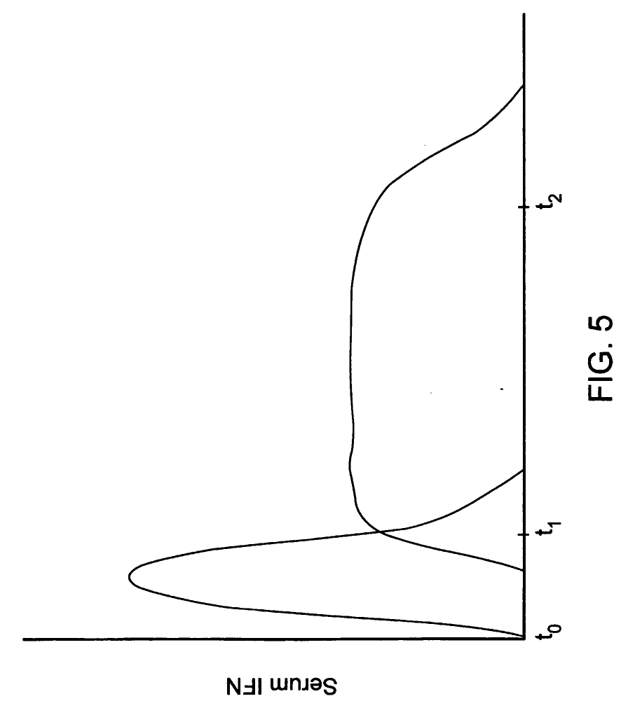

The pharmacokinetic profile illustrated in FIG. 3 can be achieved in a number of ways, including the following: (1) a composition that includes a first form of IFN-α and a second form of IFN-α is administered to an individual, where the second form of IFN-α contains a modification such that its mean residence is greater than that of the first form of IFN-α; (2) a first form of IFN-α and a second form of IFN-α are administered in separate formulations substantially simultaneously, as illustrated in FIG. 4; (3) a first form of IFN-α and a second form of IFN-α are administered in separate formulations and at separate times, as illustrated in FIG. 5.

Methods of Treating a Hepatitis Virus Infection

The instant invention provides method of treating a hepatitis virus infection. The methods generally involve administering a first form of IFN-α that has a first pharmacokinetic profile with at least a second form of IFN-α that has a second pharmacokinetic profile. The second form of IFN-α has a longer mean residence time in the body than that of the first form of IFN-α. The combination of the first and second forms of IFN-α, when administered to an individual in need of treatment with IFN-α, results in a multiphasic serum profile of IFN-α.

In the instant specification, reference to “IFN-α” without any further specific reference to a form of IFN-α (e.g., a first form or a second form of IFN-α) is meant to refer to IFN-α of any form.

In many embodiments of the invention, the methods of the invention achieve serum concentrations of antiviral agent in which the “peaks” (Cmax; the highest serum concentration of antiviral agent) and “troughs” (Cmin; the lowest serum concentration of antiviral agent) of serum antiviral agent concentration are reduced or avoided. In many embodiments, the instant methods result in Cmax:Cmin ratio of less than about 3.0, less than about 2.5, less than about 2.0, or less than about 1.5 during the second phase (e.g., during days 2-15 of treatment, during days 2-10 of treatment, during days 3-10 of treatment, or during days 3-15 of treatment, as shown in FIGS. 2-5). In some embodiments, the methods achieve a Cmax:Cmin ratio of about 1.0 during the second phase (e.g., during days 2-15 of treatment, during days 2-10 of treatment, during days 3-10 of treatment, or during days 3-15 of treatment, as shown in FIGS. 2-5).

In general, in the methods of the invention, an area under the curve (AUC) of antiviral agent serum concentration versus time during the second phase, measured during any 24-hour period of the second phase, (i.e., AUCₛᵤₛ is less than the AUC for any 24-hours period of the first phase (i.e., AUCₘᵢₙ). In other words, the AUCₛᵤₛ measured during any 24-hour period of the second phase is less than the AUCₘₐₓ measured during any 24-hour period of the first phase.

The serum concentration of antiviral agent in the first phase is effective to achieve a 1.5-log, a 2-log, a 2.5-log, a 3-log, a 3.5-log, a 4-log, a 4.5-log, or a 5-log reduction in viral titer in the serum of the individual.

The serum concentration of antiviral agent in the first phase is effective to achieve a 1.5-log, a 2-log, a 2.5-log, a 3-log, a 3.5-log, a 4-log, a 4.5-log, or a 5-log reduction in viral titer in the serum of the individual within a period of from about 12 hours to about 48 hours, or from about 16 hours to about 24 hours after the beginning of the dosing regimen.

The second concentration of antiviral agent is maintained for a period of from about 24 hours to about 48 hours, from about 2 days to about 4 days, from about 4 days to about 7 days, from about 1 week to about 2 weeks, from about 2 weeks to about 4 weeks, from about 4 weeks to about 6 weeks, from about 6 weeks to about 8 weeks, from about 8 weeks to about 12 weeks, from about 12 weeks to about 16 weeks, from about 16 weeks to about 24 weeks, or from about 24 weeks to about 48 weeks.

In the second phase, the concentration of antiviral agent in the serum is effective to reduce viral titers to undetectable levels, e.g., to about 1000 to about 5000, to about 500 to about 1000, or to about 100 to about 500 genome copies/mL serum. In some embodiments, an effective amount of antiviral agent is an amount that is effective to reduce viral load to lower than 100 genome copies/mL serum.

The serum concentration of antiviral agent in the second phase is effective to achieve a sustained viral response, e.g., no detectable HCV RNA (e.g., less than about 500, less than about 400, less than about 200, or less than about 100 genome copies per milliliter serum) is found in the patient's serum for a period of at least about one month, at least about two months, at least about three months, at least about four months, at least about five months, or at least about six months following cessation of therapy.

In some embodiments, at least a third phase follows the first and second phases. In some of these embodiments, third phase includes administering antiviral agent in a dose effective to achieve a serum concentration of antiviral agent equal or nearly equal to that of the first serum concentration. In some of these embodiments, a fourth phase includes administering antiviral agent in a dose effective to achieve a serum concentration of antiviral agent equal or nearly equal to that of the second serum concentration.

The multiphasic IFN-α serum profile is achieved by administering a first form of IFN-α and at least a second form of IFN-α that have different pharmacokinetic profiles, such that the second form of IFN-α has a longer mean residence time than that of the first form of IFN-α.

The first form of IFN-α provides for a serum concentration of IFN-α that is at or near the maximum level that is tolerable by the patient. The serum concentration that is achieved in the first phase (the first concentration) is in a range of from about 10 to about 1000, from about 10 to about 500, from about 20 to about 250, from about 30 to about 100, or from about 50 to about 75 International Units (IU)/ml. The first serum concentration is maintained for a period of from about 6 hours to about 12 hours, from about 12 hours to about 24 hours, or from about 24 hours to about 48 hours.

In the first phase, an amount of IFN-α (which can comprise a first form of IFN-α or both a first and a second form of IFN-α) is administered that is effective to achieve a serum concentration of IFN-α that is from about 65% to about 70%, from about 70% to about 75%, from about 75% to about 80%, from about 80% to about 85%, from about 85% to about 90%, from about 90% to about 95%, or from about 95% to about 100% of the maximum tolerated dose (MTD). Thus, within a period of from about 6 hours to about 12 hours, from about 12 hours to about 24 hours, or from about 24 hours to about 48 hours from the beginning of the dosing regimen, a serum concentration of IFN-α is achieved that is from about 65% to about 70%, from about 70% to about 75%, from about 75% to about 80%, from about 80% to about 85%, from about 85% to about 90%, from about 90% to about 95%, or from about 95% to about 100% of the maximum tolerated dose (MTD).

The administered dose to achieve the first serum concentration of IFN-α is in a range of from about 10 μg to about 100 μg, from about 20 μg to about 70 μg, from about 25 μg to about 60 μg, from about 30 μg to about 50 μg. These various doses refer to free interferon and the amounts of the depots to administer to achieve this will depend on drug loading efficiencies, as discussed below.

Patients with chronic hepatitis C generally have circulating virus at levels of 10⁵-10⁷ genome copies/ml. In this first phase, the serum concentration of IFN-α is effective to reduce HCV titer down to about 5×10⁴ to about 10⁵, to about 10⁴ to about 5×10⁴, or to about 5×10³ to about 10⁴ genome copies per milliliter serum.

In some embodiments, the serum concentration of IFN-α in the first phase is effective to reduce HCV titer down to about 5×10⁴ to about 10⁵, to about 10⁴ to about 5×10⁴, or to about 5×10³ to about 10⁴ genome copies per milliliter serum within a period of from about 12 hours to about 48 hours, or from about 16 hours to about 24 hours after the beginning of the dosing regimen.

In some embodiments, the serum concentration of IFN-α in the first phase is effective to achieve a 1.5-log, a 2-log, a 2.5-log, a 3-log, a 3.5-log, a 4-log, a 4.5-log, or a 5-log reduction in viral titer in the serum of the individual.

In some embodiments, the serum concentration of IFN-α in the first phase is effective to achieve a 1.5-log, a 2-log, a 2.5-log, a 3-log, a 3.5-log, a 4-log, a 4.5-log, or a 5-log reduction in viral titer in the serum of the individual within a period of from about 12 hours to about 48 hours, or from about 16 hours to about 24 hours after the beginning of the dosing regimen.

In the first phase, a serum concentration of IFN-α is achieved that is effective to reduce the viral titer to a level that is treatable with a dose of interferon that can be tolerated by an infected individual.

In the second phase, at least a second form of IFN-α is administered at a level that is effective to achieve a serum concentration of IFN-α that is well below the maximum level that can be tolerated by the patient, and that is effective to reduce the viral titer still further. In the second phase, IFN-α is administered at a dose that is effective to achieve a serum concentration of IFN-α of from about 5 IU/ml to about 50 IU/ml. In some embodiments, IFN-α is administered at a dose that is effective to achieve a serum concentration of IFN-α of from about 5 IU/ml to about 100 IU/ml or higher. In this second phase, the administered dose of IFN-α is in a range of from about 0.5×10⁶ IU to about 50×10⁶ IU.

In the second phase, at least a second form of IFN-α is administered at a level that is effective to achieve and maintain a serum concentration of IFN-α that is from about 10% to about 15%, from about 15% to about 20%, from about 20% to about 25%, from about 25% to about 30%, from about 30% to about 35%, from about 35% to about 40%, from about 40% to about 45%, or from about 45% to about 50% of the MTD. The serum concentration of IFN-α in the second phase is well below the MTD, yet effective to exert and antiviral effect. Thus, over a period of from about 48 hours to about 4 days, from about 48 hours to about 7 days, from about 48 hours to about 10 days, or from about 48 hours to about 15 days, after the beginning of the dosing regimen, a serum concentration of IFN-α is achieved (and generally maintained) that is from about 10% to about 15%, from about 15% to about 20%, from about 20% to about 25%, from about 25% to about 30%, from about 30% to about 35%, from about 35% to about 40%, from about 40% to about 45%, or from about 45% to about 50% of the MTD.

The second concentration of IFN-α is maintained for a period of from about 24 hours to about 48 hours, from about 2 days to about 4 days, from about 4 days to about 7 days, from about 1 week to about 2 weeks, from about 2 weeks to about 4 weeks, from about 4 weeks to about 6 weeks, from about 6 weeks to about 8 weeks, from about 8 weeks to about 12 weeks, from about 12 weeks to about 16 weeks, from about 16 weeks to about 24 weeks, or from about 24 weeks to about 48 weeks.

In the second phase, the second concentration of serum IFN-α is effective to reduce viral titers to about 1000 to about 5000, to about 500 to about 1000, or to about 100 to about 500 genome copies/mL serum. In some embodiments, an effective amount of IFNα is an amount that is effective to reduce viral load to lower than 100 genome copies/mL serum.

The second concentration of serum IFN-α is effective to achieve a sustained viral response, e.g., no detectable HCV RNA (e.g., less than about 500, less than about 200, or less than about 100 genome copies per milliliter serum) is found in the patient's serum for a period of at least about one month, at least about tro months, at least about three months, at least about four months, at least about five months, or at least about six months following cessation of therapy.

In some embodiments, at least a third phase follows the first and second phases. In some of these embodiments, third phase includes administering IFN-α in a dose effective to achieve a serum concentration of IFN-α equal or nearly equal to that of the first serum concentration. In some of these embodiments, a fourth phase includes administering IFN-α in a dose effective to achieve a serum concentration of IFN-α equal or nearly equal to that of the second serum concentration.

In some embodiments, a composition of the invention comprises a first form of IFN-α and at least a second form of IFN-α, wherein the first form of IFN-α is not modified to increase its mean residence time and the second form of IFN-α is modified to increase its mean residence time.

Modifications of IFN-α that increase its mean residence time in the body include, but are not limited to, conjugation of one or more moieties to the IFN-α polypeptide, which moieties include, but are not limited to, proteins, oligosaccharides, polysaccharides, and polyethylene glycol (PEG). In the embodiments discussed below, PEG-modified IFN-α is exemplified as the second form of IFN-α. However, those skilled in the art will appreciate that other modifications of IFN-α that have increased residence time relative to IFN-α lacking those modifications can also be used.

Amounts of a first form of IFN-α and a second form of IFN-α to be administered are expressed in micrograms, as described above. Alternatively, the doses are also expressed as Units or International Units (IU) of activity. Units or IU are measured in vitro as the ability of the interferon to inhibit the cytopathic effect of a suitable virus (e.g. encephalomyocarditis virus (EMC), vesicular stomatitis virus, Semliki forest virus) after infection of an appropriate cell line (e.g., the human lung carcima cell lines, A549; HEP2/C; and the like). The antiviral activity is measured against a reference standard such as human interferon alpha supplied by WHO. Such methods are detailed in numerous references, including the following: Familletti, P. C., Rubinstein, S and Pestka, S.(1981) “A convenient and rapid cytopathic effect inhibition assay for interferon”, Methods in Enzymol, Vol 78(S. Pestka, ed), Academic Press, New York pages 387-394.

The amounts of interferon administered will depend on the specific activities of the compounds and their biological performance in vivo. For example, IFN-α 2b is administered at 11.54 μg protein three times a week corresponding to 3×10⁶ IU per injection (specific activity, 2.68×10⁶ IU/mg). On the other hand, CIFN alfa-con 1 is administered at 9 μg doses per injection corresponding to 9×10⁶ IU per administration (specific activity, 1×10⁹ IU/mg). However, in view of the fact that PEGylation reactions often result in a reduction in activity, larger mass doses of PEGylated material are administered to achieve efficacy (e.g. reduction in viral load; sustained viral response, etc.).

First Form of IFN-α

The first form of IFN-α is any form of IFN-α that does not include a modification that increases its mean residence time in the body relative to naturally occurring IFN-α. Any known IFN-α can be used as the first form. The term “interferon-alpha” as used herein refers to a family of related polypeptides that inhibit viral replication and cellular proliferation and modulate immune response. The term “IFN-α” includes naturally occurring IFN-α; and synthetic IFN-α); and analogs of naturally occurring or synthetic IFN-α; essentially any IFN-α that has antiviral properties, as described for naturally occurring IFN-α.

Suitable alpha interferons include, but are not limited to, naturally-occurring IFN-α (including, but not limited to, naturally occurring IFN-α2a, IFN-α2b); recombinant interferon alpha-2b such as Intron®A interferon available from Schering Corporation, Kenilworth, N. J.; recombinant interferon alpha-2a such as Roferon® interferon available from Hoffmann-La Roche, Nutley, N. J.; recombinant interferon alpha-2C such as Berofor® alpha 2 interferon available from Boehringer Ingelheim Pharmaceutical, Inc., Ridgefield, Conn.; interferon alpha-n1, a purified blend of natural alpha interferons such as Sumiferon available from Sumitomo, Japan or as Wellferon® interferon alpha-n1 (INS) available from the Glaxo-Wellcome Ltd., London, Great Britain; and interferon alpha-n3 a mixture of natural alpha interferons made by Interferon Sciences and available from the Purdue Frederick Co., Norwalk, Conn., under the Alferon® Tradename.

The term “IFN-α” also encompasses consensus IFN-α. Consensus IFN-α (also referred to as “CIFN” and “IFN-con” and “IFN-alpha con”) encompasses but is not limited to the amino acid sequences designated IFN-con₁ (sometimes referred to as “CIFN-alpha con1,” “IFN-alpha con1,” or “IFN-con1”), IFN-con2 and IFN-con3 which are disclosed in U.S. Pat. Nos. 4,695,623 and 4,897,471; and Infergen® (Amgen, Thousand Oaks, Calif.). Consensus interferon are generally defined by determination of a consensus sequence of naturally occurring interferon alphas. DNA sequences encoding IFN-con may be synthesized as described in the aforementioned patents or other standard methods. Use of CIFN, especially Infergen, is of particular interest.

In some embodiments, the first form of IFN-α is an N-blocked species, wherein the N-terminal amino acid is acylated with an acyl group, such as a formyl group, an acetyl group, a malonyl group, and the like.

PEGylated IFN-α

Any of the above-mentioned IFN-α polypeptides can be modified with one or more polyethylene glycol moieties, i.e., PEGylated. The PEG molecule of a PEGylated IFN-α polypeptide is conjugated to one or more amino acid side chains of the IFN-α polypeptide. In some embodiments, the PEGylated IFN-α contains a PEG moiety on only one amino acid. In other embodiments, the PEGylated IFN-α contains a PEG moiety on two or more amino acids, e.g., the IFN-α contains a PEG moiety attached to two, three, four, five, six, seven, eight, nine, or ten different amino acid residues.

IFN-α may be coupled directly to PEG (i.e., without a linking group) through an amino group, a sulfhydryl group, a hydroxyl group, or a carboxyl group.

In some embodiments, the PEGylated IFN-α is PEGylated at or near the amino terminus (N-terminus) of the IFN-α polypeptide, e.g., the PEG moiety is conjugated to the IFN-α polypeptide at one or more amino acid residues from amino acid 1 through amino acid 4, or from amino acid 5 through about 10.

In other embodiments, the PEGylated IFN-α is PEGylated at one or more amino acid residues from about 10 to about 28.

In other embodiments, the PEGylated IFN-α is PEGylated at or near the carboxyl terminus (C-terminus) of the IFN-α polypeptide, e.g., at one or more residues from amino acids 156-166, or from amino acids 150 to 155.

In other embodiments, the PEGylated IFN-α is PEGylated at one or more amino acid residues at one or more residues from amino acids 100-114.

Selection of the attachment site of polyethylene glycol on the IFN-α is determined by the role of each of the sites within the receptor-binding and/or active site domains of the protein, as would be known to the skilled artisan. In general, amino acids at which PEGylation is to be avoided include amino acid residues from amino acid 30 or amino acid 40; and amino acid residues from amino acid 113 to amino acid 149.

In some embodiments, PEG is attached to IFN-α via a linking group. The linking group is any biocompatible linking group, where “biocompatible” indicates that the compound or group is non-toxic and may be utilized in vitro or in vivo without causing injury, sickness, disease, or death. PEG can be bonded to the linking group, for example, via an ether bond, an ester bond, a thiol bond or an amide bond. Suitable biocompatible linking groups include, but are not limited to, an ester group, an amide group, an imide group, a carbamate group, a carboxyl group, a hydroxyl group, a carbohydrate, a succinimide group (including, for example, succinimidyl succinate (SS), succinimidyl propionate (SPA), succinimidyl carboxymethylate (SCM), succinimidyl succinamide (SSA) or N-hydroxy succinimide (NHS)), an epoxide group, an oxycarbonylimidazole group (including, for example, carbonyldimidazole (CDI)), a nitro phenyl group (including, for example, nitrophenyl carbonate (NPC) or trichlorophenyl carbonate (TPC)), a trysylate group, an aldehyde group, an isocyanate group, a vinylsulfone group, a tyrosine group, a cysteine group, a histidine group or a primary amine. Methods for attaching a PEG to an IFN-α polypeptide are known in the art, and any known method can be used. See, for example, by Park et al, Anticancer Res., 1:373-376 (1981); Zaplipsky and Lee, Polyethylene Glycol Chemistry: Biotechnical and Biomedical Applications, J. M. Harris, ed., Plenum Press, NY, Chapter 21 (1992); and U.S. Pat. No. 5,985,265.

Pegylated IFN-α, and methods for making same, are discussed in, e.g., U.S. Pat. Nos. 5,382,657; 5,981,709; 5,985,265; and 5,951,974. Pegylated IFN-α encompasses conjugates of PEG and any of the above-described IFN-α molecules, including, but not limited to, PEG conjugated to interferon alpha-2a (Roferon, Hoffman LaRoche, Nutley, N. J.), where PEGylated Roferon is known as Pegasys (Hoffman LaRoche); interferon alpha 2b (Intron, Schering-Plough, Madison, N. J.), where PEGylated Intron is known as PEG-Intron (Schering-Plough); interferon alpha-2c (Berofor Alpha, Boehringer Ingelheim, Ingelheim, Germany); and consensus interferon (CIFN) as defined by determination of a consensus sequence of naturally occurring interferon alphas (Infergen, Amgen, Thousand Oaks, Calif.), where PEGylated Infergen is referred to as PEG-Infergen.

In some embodiments, the first form of IFN-α is N-blocked (and unPEGylated) IFN-α, and the second form of IFN-α is N-terminally PEGylated IFN-α. Approximately 30% of a bacterially-produced population of IFN-α is N-terminally blocked with an acyl group, and cannot be PEGylated at the N-terminus. N-terminal PEGylation of bacterially-produced IFN-α results in a population of about 30%-40% N-blocked (and unPEGylated) and about 60%-70% N-terminally PEGylated IFN-α. In some of these embodiments, the IFN-α is CIFN Alfacon-1. In some embodiments, the N-terminally PEGylated, bacterially-produced IFN-α is subjected to one or more separation steps to separate the PEGylated IFN-α from the unPEGylated (and N-blocked) IFN-α. Separation is achieved using any known method, including, but not limited to, size exclusion chromatography, HPLC, and the like. Once the two subpopulations, i.e., the first subpopulation of N-blocked, unPEGylated IFN-α and the second subpopulation of N-terminally PEGylated IFN-α are separated from one another, the two subpopulations are either re-mixed at defined ratios (as described herein) and administered together, or are administered separately.

In other embodiments, the first form of IFN-α is N-blocked (and unPEGylated) IFN-α, and the second form of IFN-α is C-terminally PEGylated IFN-α. For example, the N-blocked, unPEGylated IFN-α described above that is separated from PEGylated IFN-α is mixed with C-terminal PEGylated IFN-α.

Polyethylene Glycol

Polyethylene glycol suitable for conjugation to an IFN-α polypeptide is soluble in water at room temperature, and has the general formula R(O—CH₂—CH₂)ₙO—R, where R is hydrogen or a protective group such as an alkyl or an alkanol group, and where n is an integer from 1 to 1000. Where R is a protective group, it generally has from 1 to 8 carbons.

In many embodiments, PEG has at least one hydroxyl group, e.g., a terminal hydroxyl group, which hydroxyl group is modified to generate a functional group that is reactive with an amino group, e.g., an epsilon amino group of a lysine residue, a free amino group at the N-terminus of a polypeptide, or any other amino group such as an amino group of asparagine, glutamine, arginine, or histidine.

In other embodiments, PEG is derivatized so that it is reactive with free carboxyl groups in the IFN-α polypeptide, e.g., the free carboxyl group at the carboxyl terminus of the IFN-α polypeptide. Suitable derivatives of PEG that are reactive with the free carboxyl group at the carboxyl-terminus of IFN-α include, but are not limited to PEG-amine, and hydrazine derivatives of PEG (e.g., PEG-NH—NH₂).

In other embodiments, PEG is derivatized such that it comprises a terminal thiocarboxylic acid group, —COSH, which selectively reacts with amino groups to generate amide derivatives. Because of the reactive nature of the thio acid, selectivity of certain amino groups over others is achieved. For example, —SH exhibits sufficient leaving group ability in reaction with N-terminal amino group at appropriate pH conditions such that the ε-amino groups in lysine residues are protonated and remain non-nucleophilic. On the other hand, reactions under suitable pH conditions may make some of the accessible lysine residues to react with selectivity.

In other embodiments, the PEG comprises a reactive ester such as an N-hydroxy succinimidate at the end of the PEG chain. Such an N-hydroxysuccinimidate-containing PEG molecule reacts with select amino groups at particular pH conditions such as neutral 6.5-7.5. For example, the N-terminal amino groups may be selectively modified under neutral pH conditions. However, if the reactivity of the reagent were extreme, accessible-NH₂ groups of lysine may also react.

The PEG can be conjugated directly to the IFN-α polypeptide, or through a linker. In some embodiments, a linker is added to the IFN-α polypeptide, forming a linker-modified IFN-α polypeptide. Such linkers provide various functionalities, e.g., reactive groups such sulfhydryl, amino, or carboxyl groups to couple a PEG reagent to the linker-modified IFN-α polypeptide.

In some embodiments, the PEG conjugated to the IFN-α polypeptide is linear. In other embodiments, the PEG conjugated to the IFN-α polypeptide is branched. Branched PEG derivatives such as those described in U.S. Pat. No. 5,643,575, “star-PEG's” and multi-armed PEG's such as those described in Shearwater Polymers, Inc. catalog “Polyethylene Glycol Derivatives 1997-1998.” Star PEGs are described in the art including, e.g., in U.S. Pat. No. 6,046,305.

PEG having a molecular weight in a range of from about 2 kDa to about 100 kDa, is generally used, where the term “about,” in the context of PEG, indicates that in preparations of polyethylene glycol, some molecules will weigh more, some less, than the stated molecular weight. For example, PEG suitable for conjugation to IFN-α has a molecular weight of from about 2 kDa to about 5 kDa, from about 5 kDa to about 10 kDa, from about 10 kDa to about 15 kDa, from about 15 kDa to about 20 kDa, from about 20 kDa to about 25 kDa, from about 25 kDa to about 30 kDa, from about 30 kDa to about 40 kDa, from about 40 kDa to about 50 kDa, from about 50 kDa to about 60 kDa, from about 60 kDa to about 70 kDa, from about 70 kDa to about 80 kDa, from about 80 kDa to about 90 kDa, or from about 90 kDa to about 100 kDa.

Preparing PEG-IFN-α Conjugates

As discussed above, the PEG moiety can be attached, directly or via a linker, to an amino acid residue at or near the N-terminus, internally, or at or near the C-terminus of the IFN-α polypeptide. Conjugation can be carried out in solution or in the solid phase.

N-Terminal Linkage

Methods for attaching a PEG moiety to an amino acid residue at or near the N-terminus of an IFN-α polypeptide are known in the art. See, e.g., U.S. Pat. No. 5,985,265.

In some embodiments, known methods for selectively obtaining an N-terminally chemically modified IFN-α are used. For example, a method of protein modification by reductive alkylation which exploits differential reactivity of different types of primary amino groups (lysine versus the N-terminus) available for derivatization in a particular protein can be used. Under the appropriate reaction conditions, substantially selective derivatization of the protein at the N-terminus with a carbonyl group containing polymer is achieved. The reaction is performed at pH which allows one to take advantage of the pK₈ differences between the ε-amino groups of the lysine residues and that of the α-amino group of the N-terminal residue of the protein. By such selective derivatization attachment of a PEG moiety to the IFN-α is controlled: the conjugation with the polymer takes place predominantly at the N-terminus of the IFN-α and no significant modification of other reactive groups, such as the lysine side chain amino groups, occurs.

C-Terminal Linkage

N-terminal-specific coupling procedures such as described in U.S. Pat. No. 5,985,265 provide predominantly monoPEGylated products. However, the purification procedures aimed at removing the excess reagents and minor multiply PEGylated products remove the N-terminal blocked polypeptides. In terms of therapy, such processes lead to significant increases in manufacturing costs. For example, examination of the structure of the well-characterized Infergen sequence reveals that the clipping is approximate 5% at the carboxyl terminus and thus there is only one major C-terminal sequence. Thus, in some embodiments, N-terminally PEGylated IFN-α is not used; instead, the IFN-α polypeptide is C-terminally PEGylated.

An effective synthetic as well as therapeutic approach to obtain mono PEGylated Infergen product is therefore envisioned as follows:

A PEG reagent that is selective for the C-terminal can be prepared with or without spacers. For example, polyethylene glycol modified as methyl ether at one end and having an amino function at the other end may be used as the starting material.

Preparing or obtaining a water-soluble carbodiimide as the condensing agent can be carried out. Coupling IFN-α (e.g., Infergen or consensus interferon) with a water-soluble carbodiimide as the condensing reagent is generally carried out in aqueous medium with a suitable buffer system at an optimal pH to effect the amide linkage. A high molecular weight PEG can be added to the protein covalently to increase the molecular weight.

The reagents selected will depend on process optimization studies. A non-limiting example of a suitable reagent is EDAC or 1-ethyl-3-(3-dimethylaminopropyl) carbodiimide. The water solubility of EDAC allows for direct addition to a reaction without the need for prior organic solvent dissolution. Excess reagent and the isourea formed as the by-product of the cross-linking reaction are both water-soluble and may easily be removed by dialysis or gel filtration. A concentrated solution of EDC in water is prepared to facilitate the addition of a small molar amount to the reaction. The stock solution is prepared and used immediately in view of the water labile nature of the reagent. Most of the synthetic protocols in literature suggest the optimal reaction medium to be in pH range between 4.7 and 6.0. However the condensation reactions do proceed without significant losses in yields up to pH 7.5. Water may be used as solvent. In view of the contemplated use of Infergen, preferably the medium will be 2-(N-morpholino)ethane sulfonic acid buffer pre-titrated to pH between 4.7 and 6.0. However, 0.1 M phosphate in the pH 7-7.5 may also be used in view of the fact that the product is in the same buffer. The ratios of PEG amine to the IFN-α molecule is optimized such that the C-terminal carboxyl residue(s) are selectively PEGylated to yield monoPEGylated derivative(s).

Even though the use of PEG amine has been mentioned above by name or structure, such derivatives are meant to be exemplary only, and other groups such as hydrazine derivatives as in PEG-NH—NH₂ which will also condense with the carboxyl group of the IFN-α protein, can also be used. In addition to aqueous phase, the reactions can also be conducted on solid phase. Polyethylene glycol can be selected from list of compounds of molecular weight ranging from 300-40000. The choice of the various polyethylene glycols will also be dictated by the coupling efficiency and the biological performance of the purified derivative in vitro and in vivo i.e., circulation times, anti viral activities etc.

Additionally, suitable spacers can be added to the C-terminal of the protein. The spacers may have reactive groups such as SH, NH₂ or COOH to couple with appropriate PEG reagent to provide the high molecular weight IFN-α derivatives. A combined solid/solution phase methodology can be devised for the preparation of C-terminal pegylated interferons. For example, the C-terminus of IFN-α is extended on a solid phase using a Gly-Gly-Cys-NH₂ spacer and then monopegylated in solution using activated dithiopyridyl-PEG reagent of appropriate molecular weights. Since the coupling at the C-terminus is independent of the blocking at the N-terminus, the envisioned processes and products will be beneficial with respect to cost (a third of the protein is not wasted as in N-terminal PEGylation methods) and contribute to the economy of the therapy to treat chronic hepatitis C infections, liver fibrosis etc.

There may be a more reactive carboxyl group of amino acid residues elsewhere in the molecule to react with the PEG reagent and lead to monoPEGylation at that site or lead to multiple PEGylations in addition to the COOH group at the C-terminus of the IFN-α. It is envisioned that these reactions will be minimal at best owing to the steric freedom at the C-terminal end of the molecule and the steric hindrance imposed by the carbodiimides and the PEG reagents such as in branched chain molecules. It is therefore the preferred mode of PEG modification for Infergen and similar such proteins, native or expressed in a host system, which may have blocked N-termini to varying degrees to improve efficiencies and maintain higher in vivo biological activity.

Another method of achieving C-terminal PEGylation is as follows. Selectivity of C-terminal PEGylation is achieved with a sterically hindered reagent which excludes reactions at carboxyl residues either buried in the helices or internally in IFN-α. For example, one such reagent could be a branched chain PEG ˜40 kd in molecular weight and this agent could be synthesized as follows:

OH₃C—(CH₂CH₂O)n-CH₂CH₂NH₂+Glutamic Acid i.e., HOCO—CH₂CH₂CH(NH2)-COOH is condensed with a suitable agent e.g., dicyclohexyl carbodiimide or water-soluble EDC to provide the branched chain PEG agent OH₃C—(CH₂CH₂O)ₙ—CH₂CH₂NHCOCH(NH₂)CH₂OCH₃—(CH₂CH₂O)ₙ—CH₂CH₂NHCOCH₂.

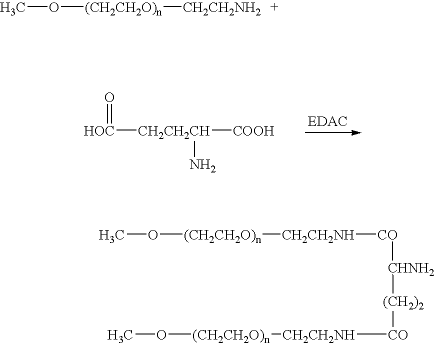

This reagent can be used in excess to couple the amino group with the free and flexible carboxyl group of IFN-α to form the peptide bond.

If desired, PEGylated IFN-α is separated from unPEGylated IFN-α using any known method, including, but not limited to, ion exchange chromatography, size exclusion chromatography, and combinations thereof. For example, where the PEG-IFN-α conjugate is a monoPEGylated IFN-α, the products are first separated by ion exchange chromatography to obtain material having a charge characteristic of monoPEGylated material (other multi-PEGylated material having the same apparent charge may be present), and then the monoPEGylated materials are separated using size exclusion chromatography.

Consensus interferon alpha con-1 (IFN-α con1) expressed in E. coli and purified has at least three N-termini: approximately one third of the bacterially-produced CIFN has an N-terminal methionine; approximately one third of the bacterially-produced CIFN has an N-terminal cysteine; and approximately one third of the bacterially-produced CIFN has either an N-terminal methionine or an N-terminal cysteine that is acylated with a number of acyl groups, including formyl, acetyl, and malonyl groups. The N-terminally acylated species are collectively referred to as N-blocked species. The N-terminally blocked protein accounts for approximately a third of the molecule on molar or mass basis. Therefore, since PEGylation does not occur at the N-terminus of the N-blocked species, PEGylating at the N-terminus of the bacterially-produced population of IFN-α is restricted in yield to approximately 60-70%.

Common method of PEGylation leads to mutiple PEGylations of amino groups in proteins and lead to losses in activity of the protein. Modification of external lysine residues results in a loss of biological activity of certain proteins. In addition, attachment of large PEGs or multiple sites of PEGylation can also result in decreased in vitro bioactivity because there is an increased chance of PEG attachment occurring at receptor-binding domains.

In addition, recent PEGylation protocols adopted for the syntheses of PEG-Intron and Pegasys suggest that the methods of internal residue modifications of histidine or lysines lead to significant reductions in the antiviral activities in vitro of the modified proteins contributing to increased costs of these products.

Selectivity in PEGylation can be achieved not only by the molar ratios of reagents to substrate but also by the choice of the polyethylene glycol of appropriate structure. For example, it is well known that branched chain PEGs attach at single or fewer sites than do linear PEGs; therefore, branched PEGs may be preferred in some embodiments. Also branched PEGs may be less likely to interfere with the biologic activity of the native molecule than would the attachment of multiple small linear chain PEGs. It is likely that the relative freedom in the carboxyl terminus of the molecule may provide sufficient flexibility for selective PEGylation of the carboxyl group using even linear PEGs of sufficiently high molecular weight. With branched PEGs, one may gain additional selectivity owing to steric hindrance. Thus the carboxyl residues in Asp or Glu located in the side chains elsewhere in the molecule in the interior of the protein will be sterically prohibited by the coupling chemistry and the reagents. Due to the delicate balance between selectivity and protection from degradation and losses in activity of modified products, pharmacodynamics of the PEGylated molecule will be carefully examined and optimized.

Three-dimensional model proposed for the human interferon-α consensus sequence suggest fair amount of flexibility in the C-terminal region. Kom et al. (1994) J. Interferon Res. 14:1-9. The absence of sequence of 156-166 in naturally occurring IFNas does not diminish activity. Fish (1992) J. Interferon Res. 12: 257-66; Levy et al. (1981) Proc. Natl. Acad. Sci. USA 78: 6186-90. An artificially truncated analog lacking 13 carboxy-terminal residues retained activity. Wetzel et al. (1982) UCLA Symp Mol. Cell Biol. 25. In the model, these sequences comprise about one-third of the carboxy-terminal end of helix E located at the bottom of the molecule adjacent to the amino terminus, distant from the receptor-recognition loops.

IFN-con 1 contains 19 amino acid differences from IFN α-2b. More than half of these changes are clustered on the C-terminal end of the molecule. Mutagenesis and antibody binding studies suggest that the residues in this region of the molecule are not important for receptor binding or biologic activity. Welter (1997) Seminar Oncol 24(3 suppl 9): 52-62. Thus there is a fair amount of flexibility for variations in residues at this segment of the polypeptide structure and it is possible that molecules monoPEGylated may retain complete activity. It is believed that the C-terminally monoPEGylated molecules of the invention retain complete or substantially coinplete receptor binding activity and other biologic activities of the corresponding parental (underivatized) IFN-α molecules.

Administering a First Form and a Second Form of IFN-α in Separate Formulations Substantially Simultaneously

In some embodiments, a multiphasic pharmacokinetic profile is achieved by administering a first and a second form of IFN-α in separate formulations substantially simultaneously. Thus, in some embodiments, the first form and the second form are administered in separate formulations and are administered within about 5 seconds to about 15 seconds, within about 15 seconds to about 30 seconds, within about 30 seconds to about 60 seconds, within about 1 minute to about 5 minutes, within about 5 minutes to about 15 minutes, within about 15 minutes to about 30 minutes, within about 30 minutes to about 60 minutes of one another.

In some embodiments, PEGylated IFN-α and unPEGylated IFN-α are administered in separate formulations and substantially simultaneously. In some embodiments, N-terminally PEGylated IFN-α and unPEGylated IFN-α are administered in separate formulations and substantially simultaneously. In some embodiments, C-terminally PEGylated IFN-α and unPEGylated IFN-α are administered in separate formulations and substantially simultaneously.

Administering a First Form and a Second Form of IFN-α in Separate Formulations at Separate Times

In some embodiments, a multiphasic pharmacokinetic profile is achieved by administering a first and a second form of IFN-α in separate formulations at separate times. Thus, in some embodiments, the first form and the second form are administered in separate formulations and the first form is administered at a time t₀, and the second form is administered at a second time t₁, where t₁-t₀ is from about 12 hours to about 16 hours, from about 16 hours to about 20 hours, from about 20 hours to about 24 hours, from about 24 hours to about 36 hours, or from about 36 hours to about 48 hours.

Administering Compositions Comprising a First and a Second Form of IFN-α

In some embodiments, a multiphasic pharmacokinetic profile is achieved by administering a first and a second form of IFN-α in the same formulation. Such formulations include compositions that include a first and a second form of IFN-α, as described in more detail below.

Compositions

The invention provides compositions comprising a PEGylated IFN-α. In some embodiments, a subject composition includes a first form of IFN-α and a second form of IFN-α, where the second form of IFN-α contains a PEG modification that increases its mean residence time relative to the first form of IFN-α, which first form does not contain such a modification. In other embodiments, a subject composition includes IFN-α that contains one or more PEG moieties at or near the carboxyl terminus (“C-terminally PEGylated IFN-α”).

In the subject compositions, the ratio of the first form to the second form is from 1:100 to about 1:50, from about 1:50 to about 1:25, from about 1:25 to about 1:10, from about 1:10 to about 1:5, from about 1:5 to about 1:1, from about 1:1 to about 1:0.5, from about 1:0.5 to about 1:0.1, from about 1:0.1 to about 1:0.05, from about 1:0.05 to about 1:0.04, from about 1:0.04 to about 1:0.02, or from about 1:0.02 to about 1:0.01.

In other embodiments of the invention, the composition comprises about from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, or from about 85% to about 90% unPEGylated IFN-α as a percentage of the total moles of PEGylated IFN-α and unPEGylated IFN-α in the composition.

In additional embodiments of the invention, the composition comprises from about 90% to about 85%, from about 85% to about 80%, from about 80% to about 75%, from about 75% to about 70%, from about 70% to about 65%, from about 65% to about 60%, from about 60% to about 55%, from about 55% to about 50%, from about 50% to about 45%, from about 45% to about 40%, from about 40% to about 35%, from about 35% to about 30%, from about 30% to about 25%, from about 25% to about 20%, from about 20% to about 15%, or from about 15% to about 10% PEGylated IFN-α as a percentage of the total moles of PEGylated IFN-α and unPEGylated IFN-α in the composition.

In some embodiments, a subject composition includes PEGylated IFN-α and unPEGylated IFN-α, where the PEGylated IFN-α contains one or more residues at or near the N-terminus that are linked, directly or indirectly, to a PEG moiety. For example, N-terminally PEGylated Infergen retains about 20% of the activity of unPEGylated Infergen. The reduced activity of N-terminally PEGylated IFN-α is used to provide a lower but longer acting pharmacodynamic effect after the unPEGylated IFN-α is eliminated. In particular embodiments, the N-terminally PEGylated IFN-α is N-terminally PEGylated Infergen.

In some embodiments, the starting material for PEGylation is bacterially produced IFN-α. Approximately 30-40% of bacterially produced IFN-α are derivatives in which the N-terminal amino group is acylated, which acylation prevents PEGylation at that amino group. In some embodiments, the first form of IFN-α is the acylated form of bacterially-produced IFN-α. Approximately 60-70% of the starting material is not acylated at the N-terminal amino group, and therefore is subject to PEGylation at the N-terminal amino group. Pegylation of bacterially produced IFN-α therefore results in PEGylation of approximately 60-70% of the IFN-α population.

In some embodiments, the first form of IFN-α is N-blocked (and unPEGylated) IFN-α, and the second form of IFN-α is N-terminally PEGylated IFN-α. Approximately 30% of a bacterially-produced population of IFN-α is N-terminally blocked with an acyl group, and cannot be PEGylated at the N-terminus. N-terminal PEGylation of bacterially-produced IFN-α results in a population of about 30%-40% N-blocked (and unPEGylated) and about 60%-70% N-terminally PEGylated IFN-α. In some of these embodiments, the IFN-α is CIFN Alfacon-1. In some embodiments, the N-terminally PEGylated, bacterially-produced IFN-α is subjected to one or more separation steps to separate the PEGylated IFN-α from the unPEGylated (and N-blocked) IFN-α. Separation is achieved using any known method, including, but not limited to, size exclusion chromatography, HPLC, and the like. Once the two subpopulations, i.e., the first subpopulation of N-blocked, unPEGylated IFN-α and the second subpopulation of N-terminally PEGylated IFN-α are separated from one another, the two subpopulations are either re-mixed at defined ratios (as described herein).

In some embodiments, a subject composition includes unPEGylated IFN-α and C-terminally PEGylated IFN-α, which may or may not be N-terminally blocked. In some of these embodiments, the C-terminally PEGylated IFN-α is monoPEGylated, e.g., the IFN-α contains only one PEG moiety at or near the C terminus.

C-terminally PEGylated IFN-α

In still other embodiments, a subject composition includes C-terminally PEGylated IFN-α. In some embodiments, C-terminally PEGylated IFN-α is monoPEGylated, i.e., the IFN-α contains a single PEG moiety attached to the carboxyl terminus of the IFN-α polypeptide. In some embodiments, the PEG linear. In other embodiments, the PEG moiety is branched.

In many embodiments, C-terminally PEGylated monoPEGylated IFN-α polypeptides of the invention retain at least about 50%, at least about 75%, at least about 80%, at least about 85%, at least about 90%, at least about 95%, or at least about 98% of at least one biological activity of the parent (unPEGylated) molecule. In some embodiments, a C-terminally PEGylated monoPEGylated IFN-α polypeptide of the invention retains from 95% to 100% of at least one biological activity of the parent (unPEGylated) molecule. Biological activities of IFN-α include, but are not limited to, binding to IFN-α receptor; inhibition in vitro of the cytopathic effect of EMC virus; inhibition of proliferation of DAUDI human lymphoblastoid B cell line in vitro; activation of 2′, 5′ oligo adenylate synthetase activity in vitro or in vivo; activation of synthesis of RNaseL; and the like.

Any known IFN-α polypeptide can be modified with a PEG moiety, where the PEG moiety is covalently attached to the carboxyl terminus of the IFN-α polypeptide. In a particular embodiment, the IFN-α is CIFN, e.g., CIFN Alfacon-1.

C-terminally PEGylated IFN-α of the invention exhibits increased in vivo residence time compared to unPEGylated IFN-α (which unPEGylated IFN-α also does not contain any other modification which increases its in vivo residence time). C-terminally PEGylated IFN-α exhibits in vivo residence time that is increased by about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 100% (or two-fold), from about 2-fold to about 3-fold, from about 3-fold to about 5-fold, from about 5-fold to about 7-fold, from about 7-fold to about 10-fold, from about 10-fold to about 15-fold, from about 15-fold to about 20-fold, from about 20-fold to about 25-fold, or from about 25-fold to about 30-fold, when compared to the parent molecule, e.g., the same IFN-α without any modifications.

Formulations

The above-discussed compositions can be formulated using well-known reagents and methods. Compositions are provided in formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, a PEGylated interferon is formulated in an aqueous buffer. Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

Treating Hepatitis with C-Terminally PEGylated IFN-α

In some embodiments, the invention provides a method of treating a hepatitis virus infection, the method involving administering C-terminally PEGylated IFN-α in an amount effective to reduce viral load.

An “effective amount” of C-terminally PEGylated IFN-α is an amount that is effective to achieve a 1.5-log, a 2-log, a 2.5-log, a 3-log, a 3.5-log, a 4-log, a 4.5-log, or a 5-log reduction in viral titer in the serum of the individual within a period of from about 12 hours to about 48 hours, from about 48 hours to about 3 days, from about 3 days to about 7 days, from about 7 days to about 2 weeks, from about 2 weeks to about 4 weeks, or from about 4 weeks to about 8 weeks, from about 8 weeks to about 12 weeks, from about 12 weeks to about 16 weeks, from about 16 weeks to about 24 weeks, or from about 24 weeks to about 48 weeks after the beginning of the dosing regimen.

C-terminally PEGylated IFN-α is administered daily, twice a week, once a week, once every two weeks, or three times a week for a period of from about 24 hours to about 48 hours, from about 2 days to about 4 days, from about 4 days to about 7 days, from about 1 week to about 2 weeks, from about 2 weeks to about 4 weeks, from about 4 weeks to about 6 weeks, from about 6 weeks to about 8 weeks, from about 8 weeks to about 12 weeks, from about 12 weeks to about 16 weeks, from about 16 weeks to about 24 weeks, or from about 24 weeks to about 48 weeks.

In some embodiments, C-terminally PEGylated IFN-α is administered in an amount and for a period of time effective to reduce viral titers to undetectable levels, e.g., to about 1000 to about 5000, to about 500 to about 1000, or to about 100 to about 500 genome copies/mL serum. In some embodiments, an effective amount of antiviral agent is an amount that is effective to reduce viral load to lower than 100 genome copies/mL serum.

In some embodiments, C-terminally PEGylated IFN-α is administered in a dosage of 30 μg to about 300 μg. In some embodiments, C-terminally PEGylated IFN-α is administered in doses of 32.5 μg, 65 μg, 97.5 μg, 130 μg or 162.5 μg.

In some embodiments, C-terminally PEGylated IFN-α is administered in a combination therapy, e.g., another anti-viral agent or other therapeutic agent is administered: (1) substantially simultaneously and in a separate formulation; (2) substantially simultaneously and in the same formulation; or (3) in separate formulations, and at separate times. Combination therapies are discussed in detail above.

Treatment Methods

The instant invention provides methods of treating a hepatitis virus infection. The methods generally involve administering a composition of the invention at a level and in a manner effective to achieve a multiphasic serum concentration of the antiviral agent.

In many embodiments of the invention, the dosing regimens of the methods of the invention achieve serum concentrations of antiviral agent in which the “peaks” (Cmax; the highest serum concentration of antiviral agent) and “troughs” (Cmin; the lowest serum concentration of antiviral agent) of serum antiviral agent concentration are reduced or avoided. In many embodiments, the dosing regimens of the instant methods result in Cmax:Cmin ratio of less than about 3.0, less than about 2.5, less than about 2.0, or less than about 1.5 during the second phase (e.g., during days 2-15 of treatment, during days 2-10 of treatment, during days 3-10 of treatment, or during days 3-15 of treatment, as shown in FIGS. 2-5). In some embodiments, the dosing regimens achieve a Cmax:Cmin ratio of about 1.0 during the second phase (e.g., during days 2-15 of treatment, during days 2-10 of treatment, during days 3-10 of treatment, or during days 3-15 of treatment, as shown in FIGS. 2-5).

In general, in the dosing regimens of the methods of the invention, an area under the curve (AUC) of antiviral agent serum concentration versus time during the second phase, measured during any 24-hour period of the second phase, (i.e., AUCₛᵤₛ is less than the AUC for any 24-hours period of the first phase (i.e., AUCₘₐₓ). In other words, the AUCₘₐₓ measured during any 24-hour period of the second phase is less than the AUCₘₐₓ measured during any 24-hour period of the first phase.

The serum concentration of antiviral agent in the first phase is effective to achieve a 1.5-log, a 2-log, a 2.5-log, a 3-log, a 3.5-log, a 4-log, a 4.5-log, or a 5-log reduction in viral titer in the serum of the individual.

The compositions and/or dosing regimens of the invention deliver to the patient a mixture of unPEGylated and PEGylated IFN-α that is designed to achieve an IFN-α serum concentration profile in the second phase that remains relatively constant for a period of from about 24 hours to about 48 hours, from about 2 days to about 4 days, from about 4 days to about 7 days, from about 1 week to about 2 weeks, or from about 2 weeks to about 4 weeks. In one embodiment, the composition and/or dosing regimens of the invention are designed to achieve the IFN-α serum concentration (in International Units of IFN-α per milliliter of serum (IU/ml)) profile depicted in FIG. 3, 4 or 5, wherein t₁-t₀ is from about 12 hours to about 16 hours, or from about 16 hours to about 20 hours, or from about 20 hours to about 24 hours, or from about 24 hours to about 36 hours, or from about 36 hours to about 48 hours, and t₂-t₁ is from about 24 hours to about 48 hours, from about 2 days to about 4 days, from about 4 days to about 7 days, from about 1 week to about 2 weeks, or from about 2 weeks to about 4 weeks.

The multiphasic pharmacokinetic profile depicted in FIG. 3 can be achieved by administering to a patient a pharmaceutical formulation comprising a mixture of unPEGylated and PEGylated IFN-α. The molar ratio of unPEGylated IFN-α to PEGylated IFN-α is preselected to achieve an initial peak in the total serum concentration of IFN-α (in IU/ml) at 16-24 hours following administration of drug. In embodiments of the invention exhibiting the pharmacokinetic profile depicted in FIG. 3, the area under the curve (AUCmax) of total serum concentration of IFN-α (in IU/ml) as a function of time for any 24 hour period of the first phase (t₁-t₀) is approximately two-fold larger than the AUC for any 24 hour period during the second phase (t₂-t₁) (AUCₛᵤₛ). In these embodiments, the desired 2:1 ratio of AUCmax:AUCsus can be achieved with a composition characterized by a 1:1 molar ratio of unPEGylated IFN-α:PEGYlated IFN-α provided that the unPEGylated IFN-α and the PEGylated IFN-α possess essentially the same specific activity (IU/mg of protein). In such embodiments, a composition comprising 50% unPEGylated IFN-α and 50% PEGylated IFN-α by amino acid weight percent can be administered to a patient and effect the pharmacokinetic profile of FIG. 3.

In other embodiments, the invention provides a mixture of PEGylated IFN-α and unPEGylated IFN-α in which the PEGylated IFN-α possesses a lower specific activity (in IU/mg of protein) than the underivatized, parental IFN-α. In one example, the PEGylated interferon-α-2a active ingredient of the Pegasys product exhibits approximately 10% of the specific activity (in IU/mg protein) of the unPEGylated interferon-α-2a active ingredient of the Roferon product. In another example, the PEGylated interferon-α-2b active ingredient of the PEG-Intron product exhibits approximately 10% of the specific activity (in IU/mg protein) of the unPEGylated interferon-α-2b active ingredient of the Intron-A product. In yet another example, the N-terminally monoPEGylated CIFN described in U.S. Pat. No. 5,985,265 exhibits approximately 20% of the specific activity (in IU/mg protein) of the unPEGylated CIFN active ingredient of the Infergen® Alfacon-1 product.

In embodiments that employ mixtures wherein the PEGylated IFN-α component possesses a reduced specific activity (in IU/mg protein) compared to the unPEGylated IFN-α component, the molar ratio of the unPEGylated IFN-α:PEGylated IFN-α is approximately 1:1/(percent of the unPEGylated IFN-α specific activity that is exhibited by the PEGylated IFN-α in the mixture/100. For example, the three compositions of PEGylated and unPEGylated IFN-α described above are formulated using the molar ratios shown in Table 1 below in order to effect the pharmacokinetic profile depicted in FIG. 3.

TABLE 1

| | | Molar |
|---|---|---|
| unPEGylated IFN-α. (1) | PEGylated IFN-α. (2) | Ratio (1):(2) |
| Intron-A | PEG-Intron | 1:10 |
| Roferon | Pegasys | 1:10 |
| Infergen | Infergen (N-terminally | 1:5 |
| | MonoPEGylated) | |

The serum concentration of antiviral agent in the first phase is effective to achieve a 1.5-log, a 2-log, a 2.5-log, a 3-log, a 3.5-log, a 4-log, a 4.5-log, or a 5-log reduction in viral titer in the serum of the individual within a period of from about 12 hours to about 48 hours, or from about 16 hours to about 24 hours after the beginning of the dosing regimen.

The second concentration of antiviral agent is maintained for a period of from about 24 hours to about 48 hours, from about 2 days to about 4 days, from about 4 days to about 7 days, from about 1 week to about 2 weeks, from about 2 weeks to about 4 weeks, from about 4 weeks to about 6 weeks, from about 6 weeks to about 8 weeks, from about 8 weeks to about 12 weeks, from about 12 weeks to about 16 weeks, from about 16 weeks to about 24 weeks, or from about 24 weeks to about 48 weeks.

In the second phase, the concentration of antiviral agent in the serum is effective to reduce viral titers to undetectable levels, e.g., to about 1000 to about 5000, to about 500 to about 1000, or to about 100 to about 500 genome copies/mL serum. In some embodiments, an effective amount of antiviral agent is an amount that is effective to reduce viral load to lower than 100 genome copies/mL serum.

The serum concentration of antiviral agent in the second phase is effective to achieve a sustained viral response, e.g., no detectable HCV RNA (e.g., less than about 500, less than about 200, or less than about 100 genome copies per milliliter serum) is found in the patient's serum for a period of at least about one month, at least about two months, at least about three months, at least about four months, at least about five months, or at least about six months following cessation of therapy.

In some embodiments, at least a third phase follows the first and second phases. In some of these embodiments, third phase includes administering antiviral agent in a dose effective to achieve a serum concentration of antiviral agent equal or nearly equal to that of the first serum concentration. In some of these embodiments, a fourth phase includes administering antiviral agent in a dose effective to achieve a serum concentration of antiviral agent equal or nearly equal to that of the second serum concentration.

Combination Therapies

In some embodiments, the methods provide for combination therapy comprising administering a composition of the invention and an additional therapeutic agent such as IFN-γ and/or ribavirin. In many embodiments in which the dosing regimen comprises administration of IFN-α and an additional agent such as IFN-γ and/or ribavirin, IFN-α is administered such that a multiphasic serum concentration of IFN-α is achieved, as described above.

In some embodiments, the additional therapeutic agent(s) is administered during the entire course of IFN-α treatment, and the beginning and end of the treatment periods coincide. In other embodiments, the additional therapeutic agent(s) is administered for a period of time that is overlapping with that of the IFN-α treatment, e.g., treatment with the additional therapeutic agent(s) begins before the IFN-α treatment begins and ends before the IFN-α treatment ends; treatment with the additional therapeutic agent(s) begins after the IFN-α treatment begins and ends after the IFN-γ treatment ends; treatment with the additional therapeutic agent(s) begins after the IFN-α treatment begins and ends before the IFN-α treatment ends; or treatment with the additional therapeutic agent(s) begins before the IFN-α treatment begins and ends after the IFN-α treatment ends.

In still other embodiments, the additional therapeutic agent(s) is administered before the IFN-α treatment begins, and ends once IFN-α treatment begins, e.g., the additional therapeutic agent is used in a “priming” dosing regimen.

Interferon-Gamma

The nucleic acid sequences encoding IFN-γ polypeptides may be accessed from public databases, e.g., Genbank, journal publications, etc. While various mammalian IFN-γ polypeptides are of interest, for the treatment of human disease, generally the human protein will be used. Human IFN-γ coding sequence may be found in Genbank, accession numbers X13274; V00543; and NM_{—}000619. The corresponding genomic sequence may be found in Genbank, accession numbers J00219; M37265; and V00536. See, for example. Gray et al. (1982) Nature 295:501 (Genbank X13274); and Rinderknecht et al. (1984) J.B.C. 259:6790.

An exemplary form of IFN-γ of interest is Actimmune® (human interferon) which is a single-chain polypeptide of 140 amino acids having an N-terminal methionine. It is made recombinantly in E. coli and is unglycosylated. Rinderknecht et al. (1984) J. Biol. Chem. 259:6790-6797.

The IFN-γ to be used in the methods of the present invention may be any of natural IFN-γs, recombinant IFN-γs and the derivatives thereof so far as they have an IFN-γ activity, particularly human IFN-γ activity. Human IFN-γ exhibits the antiviral and anti-proliferative properties characteristic of the interferons, as well as a number of other immunomodulatory activities, as is known in the art. Although IFN-γ is based on the sequences as provided above, the production of the protein and proteolytic processing can result in processing variants thereof. The unprocessed sequence provided by Gray et al., supra, consists of 166 amino acids (aa). Although the recombinant IFN-γ produced in E. coli was originally believed to be 146 amino acids, (commencing at amino acid 20) it was subsequently found that native human IFN-γ is cleaved after residue 23, to produce a 143 aa protein, or 144 aa if the terminal methionine is present, as required for expression in bacteria. During purification, the mature protein can additionally be cleaved at the C terminus after reside 162 (referring to the Gray et al. sequence), resulting in a protein of 139 amino acids, or 140 amino acids if the initial methionine is present, e.g. if required for bacterial expression. The N-terminal methionine is an artifact encoded by the mRNA translational “start” signal AUG that, in the particular case of E. coli expression is not processed away. In other microbial systems or eukaryotic expression systems, methionine may be removed.

For use in the subject methods, any of the native IFN-γ peptides, modifications and variants thereof, or a combination of one or more peptides may be used. IFN-γ peptides of interest include fragments, and can be variously truncated at the carboxy terminal end relative to the full sequence. Such fragments continue to exhibit the characteristic properties of human gamma interferon, so long as amino acids 24 to about 149 (numbering from the residues of the unprocessed polypeptide) are present. Extraneous sequences can be substituted for the amino acid sequence following amino acid 155 without loss of activity. See, for example, U.S. Pat. No. 5,690,925. Native IFN-γ moieties include molecules variously extending from amino acid residues 24-150; 24-151, 24-152; 24-153, 24-155; and 24-157. Any of these variants, and other variants known in the art and having IFN-γ activity, may be used in the present methods.

The sequence of the IFN-γ polypeptide may be altered in various ways known in the art to generate targeted changes in sequence. A variant polypeptide will usually be substantially similar to the sequences provided herein, i.e., will differ by at least one amino acid, and may differ by at least two but not more than about ten amino acids. The sequence changes may be substitutions, insertions or deletions. Scanning mutations that systematically introduce alanine, or other residues, may be used to determine key amino acids. Specific amino acid substitutions of interest include conservative and non-conservative changes. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that may or may not alter the primary amino acid sequence include chemical derivatization of polypeptides, e.g., acetylation, or carboxylation; changes in amino acid sequence that introduce or remove a glycosylation site; changes in amino acid sequence that make the protein susceptible to PEGylation; and the like. Also included are modifications of glycosylation, e.g., those made by modifying the glycosylation patterns of a polypeptide during its synthesis and processing or in further processing steps; e.g., by exposing the polypeptide to enzymes that affect glycosylation, such as mammalian glycosylating or deglycosylating enzymes. Also embraced are sequences that have phosphorylated amino acid residues, e.g., phosphotyrosine, phosphoserine, or phosphothreonine.

Included in the subject invention are polypeptides that have been modified using ordinary chemical techniques so as to improve their resistance to proteolytic degradation, to optimize solubility properties, or to render them more suitable as a therapeutic agent. For examples, the backbone of the peptide may be cyclized to enhance stability (see Friedler et al. (2000) J. Biol. Chem. 275:23783-23789). Analogs may be used that include residues other than naturally occurring L-amino acids, e.g., D-amino acids or non-naturally occurring synthetic amino acids. The protein may be PEGylated to enhance stability.

The polypeptides may be prepared by in vitro synthesis, using conventional methods as known in the art, by recombinant methods, or may be isolated from cells induced or naturally producing the protein. The particular sequence and the manner of preparation will be determined by convenience, economics, purity required, and the like. If desired, various groups may be introduced into the polypeptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

Ribavirin and Other Antiviral Agents

Ribavirin, 1-β-D-ribofuranosyl-1H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. The invention also contemplates use of derivatives of ribavirin (see, e.g., U.S. Pat. No. 6,277,830). Ribavirin is administered in dosages of about 400, about 800, or about 1200 mg per day.

Other antiviral agents can be delivered in the treatment methods of the invention. For example, compounds that inhibit inosine monophosphate dehydrogenase (IMPDH) may have the potential to exert direct anti viral activity, and such compounds can be administered in combination with an IFN-α composition, as described herein. Drugs that are effective inhibitors of hepatitis C NS3 protease may be administered in combination with an IFN-α composition, as described herein. Hepatitis C NS3 protease inhibitors inhibit viral replication. Other agents such as inhibitors of HCV NS3 helicase are also attractive drugs for combinational therapy, and are contemplated for use in combination therapies described herein. Ribozymes such as Heptazyme™ and phosphorothioate oligonucleotides which are complementary to HCV protein sequences and which inhibit the expression of viral core proteins are also suitable for use in combination therapies described herein.

Liver Targeting Systems

Antiviral agents described herein can be targeted to the liver, using any known targeting means. Those skilled in the art are aware of a wide variety of compounds that have been demonstrated to target compounds to hepatocytes. Such liver targeting compounds include, but are not limited to, asialoglycopeptides; basic polyamino acids conjugated with galactose or lactose residues; galactosylated albumin; asialoglycoprotein-poly-L-lysine) conjugates; lactosaminated albumin; lactosylated albumin-poly-L-lysine conjugates; galactosylated poly-L-lysine; galactose-PEG-poly-L-lysine conjugates; lactose-PEG-poly-L-lysine conjugates; asialofetuin; and lactosylated albumin.

In some embodiments, a liver targeting compound is conjugated directly to the antiviral agent. In other embodiments, a liver targeting compound is conjugated indirectly to the antiviral agent, e.g., via a linker. In still other embodiments, a liver targeting compound is associated with a delivery vehicle, e.g., a liposome or a microsphere, forming a hepatocyte targeted delivery vehicle, and the antiviral agent is delivered using the hepatocyte targeted delivery vehicle.

The terms “targeting to the liver” and “hepatocyte targeted” refer to targeting of an antiviral agent to a hepatocyte, such that at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or more, of the antiviral agent administered to the subject enters the liver via the hepatic portal and becomes associated with (e.g., is taken up by) a hepatocyte.

Formulations

The above-discussed antiviral agents can be formulated using well-known reagents and methods. Antiviral agents are provided in formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In the subject methods, the active agents may be administered to the host using any convenient means capable of resulting in the desired therapeutic effect. Thus, the agents can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of the agents can be achieved in various ways, including oral, buccal, rectal, parenteral, intraperitoneal, intradermal, transdermal, intracheal, etc.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Effective dosages of PEGylated IFN-α2a range from 90 to 180 μg per dose. Effective dosages of PEGylated IFN-α2b range from 0.5 μg/kg body weight to 1.5 μg/kg body weight per dose.

Kits with unit doses of the active agent, e.g. in oral or injectable doses, are provided. In such kits, in addition to the containers containing the unit doses will be an informational package insert describing the use and attendant benefits of the drugs in treating hepatitis. Preferred agents and unit doses are those described herein above.

Drug Delivery Systems

Any known delivery system can be used in the present invention. In addition, a combination of any known delivery system can be used.

The drug delivery system can be any device, including an implantable device, which device can be based on, for example, mechanical infusion pumps, electromechanical infusion pumps, depots, microspheres. Essentially, any drug delivery system that provides for controlled release as described above (at least biphasic release) is suitable for use in the instant invention. In some embodiments, the drug delivery system is a depot. In other embodiments, the drug delivery system is a continuous delivery device (e.g., an injectable system, a pump, etc.). In still other embodiments, t eh drug delivery system is a combination of a injection device (e.g., a syringe and needle) and a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

In some embodiments, the delivery system is a depot system. Depot systems comprise a matrix in which the IFN-α or other antiviral agent is embedded. The matrix is a polymeric or non-polymeric substance.

In certain embodiments, drug delivery system comprises a depot. The depot can comprise a homogeneous mixture of a first form of IFN-α and a second form of IFN-α. Alternatively, the depot can be “layered,” e.g., configured such that a first form of IFN-α is released, then a second form of IFN-α is released.

In some embodiments, the depot comprises a polymeric matrix. For example, a polymeric matrix derived from copolymeric and homopolymeric polyesters having hydrolysable ester linkages may be used. A number of these are known in the art to be biodegradable and to lead to degradation products having no or low toxicity. Non-limiting examples of such polymers are polyglycolic adds (PGA) and polylactic acids (PLA), poly(DL-lactic acid-co-glycolic acid)(DL PLGA), poly(D-lactic acid-coglycolic acid)(D PLGA) and poly(L-lactic acid-co-glycolic acid)(L PLGA). Exemplary ratios for lactic acid and glycolic acid polymers in poly(lactic acid-co-glycolic acid) is in the range of 100:0 (i.e. pure polylactide) to 50:50. Other useful biodegradable or bioerodable polymers include but are not limited to such polymers as poly(ε-caprolactone), poly(ε-caprolactone-CO-lactic add), poly (ε-caprolactone-CO-glycolic acid), poly(β-hydroxy butyric acid), poly(alkyl-2-cyanoacrilate), hydrogels such as poly(hydroxyethyl methacrylate), polyamides, poly(amino acids) (i.e. L-leucine, glutamic acid, L-aspartic acid and the like), poly (ester urea), poly (2-hydroxyethyl DL-aspartamide), polyacetal polymers, polyorthoesters, polycarbonate, polymaleamides, polysaccharides and copolymers thereof.

In some embodiments, the drug delivery system is a poly (lactic acid-co-glycolic acid) system. Such systems are described in the literature, e.g., in U.S. Pat. Nos. 6,183,781; and 5,654,008.

In some of these embodiments, the depot is a high viscosity liquid such as a non-polymeric non-water-soluble liquid carrier material, e.g., Sucrose Acetate Isobutyrate (SAIB) or another compound such as a compound described in U.S. Pat. Nos. 5,968,542; and 5,747,058. For example, the SABER™ system (Southern Biosystems, Inc.) is used.

Release modifying agents and/or additives can be included in the depot matrix. The term “release modifying agent”, as used herein, refers to a material which, when incorporated into a polymer/drug matrix, modifies the drug-release characteristics of the matrix. A release modifying agent can, for example, either decrease or increase the rate of drug release from the matrix. One group of release modifying agents includes metal-containing salts.

One category of additives includes biodegradable polymers and oligomers. The polymers can be used to alter the release profile of the substance to be delivered, to add integrity to the composition, or to otherwise modify the properties of the composition. Non-limiting examples of suitable biodegradable polymers and oligomers include: poly(lactide), poly(lactide-co-glycolide), poly(glycolide), poly(caprolactone), polyamides, polyanhydrides, polyamino acids, polyorthoesters, polycyanoacrylates, poly(phosphazines), poly(phosphoesters), polyesteramides, polydioxanones, polyacetals, polyketals, polycarbonates, polyorthocarbonates, degradable polyurethanes, polyhydroxybuty-ates, polyhydroxyvalerates, polyalkylene oxalates, polyalkylene succinates, poly(malic acid), chitin, chitosan, and copolymers, terpolymers, oxidized cellulose, or combinations or mixtures of the above materials.

Examples of poly(α-hydroxy acid) s include poly(glycolic acid), poly(DL-lactic acid) and poly(L-lactic acid), and their copolymers. Examples of polylactones include poly(ε-caprolactone), poly(δ-valerolactone) and poly(γ-butyrolactone).

Other additives include non-biodegradable polymers. Non-limiting examples of non-erodible polymers which can be used as additives include: polyacrylates, ethylene-vinyl acetate polymers, cellulose and cellulose derivatives, acyl substituted cellulose acetates and derivatives thereof, non-erodible polyurethanes, polystyrenes, polyvinyl chloride, polyvinyl fluoride, poly(vinyl imidazole), chlorosulphonated polyolefins, and polyethylene oxide.

A further class of additives which can be used in the present compositions are natural and synthetic oils and fats. Oils derived from animals or from plant seeds of nuts typically include glycerides of the fatty acids, chiefly oleic, palmitic, stearic, and linolenic.

Other additives include film property modifying agents and release controlling agents. Examples of film property modifying agents include plasticizers, e.g. triethyl-citrate, triacetin, polyethyleneglycol, polyethyleneoxide etc. Examples of release-controlling agents include inorganic bases (e.g. sodium hydroxide, potassium hydroxide, sodium carbonate, potassium carbonate, etc), organic bases (e.g. ethanol amine, diethanole amine, triethanole amine, lidocaine, tetracaine, etc,), inorganic acids (e.g. ammonium sulfate, ammonium chloride, etc), organic acids (e.g. citric acid, lactic acid, glycolic acid, ascorbic acid, etc), and solid soluble substances which upon release create pores in the coating (e.g. crystals of sodium chloride, glucose, mannitol, sucrose, etc).

In some embodiments, the drug delivery system is a polyethylene glycol-poly(lactic co-glycolic) acid (PEG-PLGA)-based aqueous injectible thermosensitive gel, as described in, e.g., U.S. Pat. Nos. 6,201,071; 6,117,949; and 6,004,573. For example, the depot can comprise a water soluble, biodegradable ABA- or BAB-type tri-block polymer is disclosed that is made up of a major amount of a hydrophobic A polymer block made of a biodegradable polyester and a minor amount of a hydrophilic PEG B polymer block, having an overall average molecular weight of between about 2000 and 4990, and that possesses reverse thermal gelation properties. Such materials form a gel depot within the body, from which the drugs are released at a controlled rate.

In some embodiments, the drug delivery system is a polyamino acid-based system, e.g., as described in U.S. Pat. Nos. 6,071,538; 6,245,359; 6,221,367; and 6,099,856.

In other embodiments, the drug delivery system is a microsphere. Microspheres are amply described in the literature.

In another embodiments, the drug delivery system is a pump, e.g., an implantable pump, particularly an adjustable implantable pump. Of particular interest is the use of an adjustable pump, particularly a pump that is adjustable while in position for delivery (e.g., externally adjustable from outside the patient's body. Such pumps include programmable pumps that are capable of providing high concentrations of IFN-α or other antiviral agent over extended periods of time, e.g., 24-72 hours, and to achieve AUC serum IFN-α concentrations to be therapeutically effective.

In some embodiments, the delivery device is a Medipad® device (Elan Pharm Int'l. Ltd.).

Mechanical or electromechanical infusion pumps can also be suitable for use with the present invention. Examples of such devices include those described in, for example, U.S. Pat. Nos. 4,692,147; 4,360,019; 4,487,603; 4,360,019; 4,725,852, and the like. In general, the present methods of drug delivery can be accomplished using any of a variety of refillable, pump systems. Pumps provide consistent, controlled release over time.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to a subdermal, subcutaneous, intramuscular, or other suitable site within a subject's body. Subcutaneous implantation sites are generally preferred because of convenience in implantation and removal of the drug delivery device.

As noted above, a combination of delivery systems can be used. As one non-limiting example, a PLGA based system which has an initial drug release or burst characteristic is combined with a sucrose acetate isobutyrate based system with no drug release as a burst may be combined together to achieve the desired profiles taught by this invention. As another non-limiting example, a loading dose such as a bolus followed by a zero-order throughput as realized or achieved with a device system. The delivery molecule may be an alpha interferon or a PEG derivatized alpha interferon with all these delivery systems.

Depending on the drug delivery system, IFN-α can be administered orally, subcutaneously, intramuscularly, parenterally, or by other routes such as transdermally, cutaneously, etc. There could be a burst of the drug when administered by such routes e.g., orally except that the drug enters portal circulation as in oral delivery and therefore of utility in targeting the drug to the desired organ, namely liver.

In many embodiments, the IFN-α is delivered subcutaneously.

IFN-α is administered to individuals in a formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

IFN-α can be administered together with (i.e., simultaneously in separate formulations; simultaneously in the same formulation; administered in separate formulations and within about 48 hours, within about 36 hours, within about 24 hours, within about 16 hours, within about 12 hours, within about 8 hours, within about 4 hours, within about 2 hours, within about 1 hour, within about 30 minutes, or within about 15 minutes or less) one or more additional therapeutic agents.

In other embodiments, patients are treated with a combination of IFN-α and ribavirin. Ribavirin, 1-β-D-ribofuranosyl-1H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. The ribavirin may be administered orally in capsule or tablet form in association with the administration of IFN-α. Of course, other types of administration of both medicaments, as they become available are contemplated, such as by nasal spray, transdermally, intravenous, by suppository, by sustained release dosage form, etc. Any form of administration will work so long as the proper dosages are delivered without destroying the active ingredient. If administered, ribavirin is administered in an amount ranging from about 400 mg to about 1200 mg, from about 600 mg to about 1000 mg, or from about 700 to about 900 mg per day.

In some embodiments, the combination therapy comprises IFN-α and IFN-γ. In some of these embodiments, IFN-α and IFN-γ are administered in the same formulation, and are administered simultaneously. In other embodiments, IFN-α and IFN-γ are administered separately, e.g., in separate formulations. In some of these embodiments, IFN-α and IFN-γ are administered separately, and are administered simultaneously. In other embodiments, IFN-α and IFN-γ are administered separately and are administered within about 5 seconds to about 15 seconds, within about 15 seconds to about 30 seconds, within about 30 seconds to about 60 seconds, within about 1 minute to about 5 minutes, within about 5 minutes to about 15 minutes, within about 15 minutes to about 30 minutes, within about 30 minutes to about 60 minutes, within about 1 hour to about 2 hours, within about 2 hours to about 6 hours, within about 6 hours to about 12 hours, within about 12 hours to about 24 hours, or within about 24 hours to about 48 hours of one another.

Determining Effectiveness of Treatment

Whether a subject method is effective in treating a hepatitis virus infection, particularly an HCV infection, can be determined by measuring viral load, or by measuring a parameter associated with HCV infection, including, but not limited to, liver fibrosis.

Viral load can be measured by measuring the titer or level of virus in serum. These methods include, but are not limited to, a quantitative polymerase chain reaction (PCR) and a branched DNA (bDNA) test. For example, quantitative assays for measuring the viral load (titer) of HCV RNA have been developed. Many such assays are available commercially, including a quantitative reverse transcription PCR (RT-PCR) (Amplicor HCV Monitor™, Roche Molecular Systems, New Jersey); and a branched DNA (deoxyribonucleic acid) signal amplification assay (Quantiplex™ HCV RNA Assay (bDNA), Chiron Corp., Emeryville, Calif.). See, e.g., Gretch et al. (1995) Ann. Intern. Med. 123:321-329.

As noted above, whether a subject method is effective in treating a hepatitis virus infection, e.g., an HCV infection, can be determined by measuring a parameter associated with hepatitis virus infection, such as liver fibrosis. Liver fibrosis reduction is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

As one non-limiting example, levels of serum alanine aminotransferase (ALT) are measured, using standard assays. In general, an ALT level of less than about 45 international units per milliliter serum is considered normal. In some embodiments, an effective amount of IFNα is an amount effective to reduce ALT levels to less than about 45 IU/ml serum.

Methods of Treating Liver Fibrosis

The present invention provides methods of treating liver fibrosis. The methods involve administering an antiviral agent, as describe above, wherein viral load is reduced in the individual, and wherein liver fibrosis is treated. Treating liver fibrosis includes reducing the risk that liver fibrosis will occur; reducing a symptom associated with liver fibrosis; and increasing liver function.

Whether treatment with antiviral agent as described herein is effective in reducing liver fibrosis is determined by any of a number of well-established techniques for measuring liver fibrosis and liver function. Liver fibrosis reduction is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems.

The METAVIR scoring system is based on an analysis of various features of a liver biopsy, including fibrosis (portal fibrosis, centrilobular fibrosis, and cirrhosis); necrosis (piecemeal and lobular necrosis, acidophilic retraction, and ballooning degeneration); inflammation (portal tract inflammation, portal lymphoid aggregates, and distribution of portal inflammation); bile duct changes; and the Knodell index (scores of periportal necrosis, lobular necrosis, portal inflammation, fibrosis, and overall disease activity). The definitions of each stage in the METAVIR system are as follows: score: 0, no fibrosis; score: 1, stellate enlargement of portal tract but without septa formation; score: 2, enlargement of portal tract with rare septa formation; score: 3, numerous septa without cirrhosis; and score: 4, cirrhosis.

Knodell's scoring system, also called the Hepatitis Activity Index, classifies specimens based on scores in four categories of histologic features: I. Periportal and/or bridging necrosis; II. Intralobular degeneration and focal necrosis; III. Portal inflammation; and IV. Fibrosis. In the Knodell staging system, scores are as follows: score: 0, no fibrosis; score: 1, mild fibrosis (fibrous portal expansion); score: 2, moderate fibrosis; score: 3, severe fibrosis (bridging fibrosis); and score: 4, cirrhosis. The higher the score, the more severe the liver tissue damage. Knodell (1981) Hepatol. 1:431.

In the Scheuer scoring system scores are as follows: score: 0, no fibrosis; score: 1, enlarged, fibrotic portal tracts; score: 2, periportal or portal-portal septa, but intact architecture; score: 3, fibrosis with architectural distortion, but no obvious cirrhosis; score: 4, probable or definite cirrhosis. Scheuer (1991) J. Hepatol. 13:372.

The Ishak scoring system is described in Ishak (1995) J. Hepatol. 22:696-699. Stage 0, No fibrosis; Stage 1, Fibrous expansion of some portal areas, with or without short fibrous septa; stage 2, Fibrous expansion of most portal areas, with or without short fibrous septa; stage 3, Fibrous expansion of most portal areas with occasional portal to portal (P-P) bridging; stage 4, Fibrous expansion of portal areas with marked bridging (P-P) as well as portal-central (P-C); stage 5, Marked bridging (P-P and/or P-C) with occasional nodules (incomplete cirrhosis); stage 6, Cirrhosis, probable or definite. The benefit of anti-fibrotic therapy can also be measured and assessed by using the Child-Pugh scoring system which comprises a multicomponent point system based upon abnormalities in serum bilirubin level, serum albumin level, prothrombin time, the presence and severity of ascites, and the presence and severity of encephalopathy. Based upon the presence and severity of abnormality of these parameters, patients may be placed in one of three categories of increasing severity of clinical disease: A, B, or C.

In some embodiments, a therapeutically effective amount of antiviral agent is an amount of antiviral agent that effects a change of one unit or more in the fibrosis stage based on pre- and post-therapy liver biopsies. In particular embodiments, a therapeutically effective amount of IFN-α and IFN-γ reduces liver fibrosis by at least one unit in the METAVIR, the Knodell, the Scheuer, the Ludwig, or the Ishak scoring system.

Secondary, or indirect, indices of liver function can also be used to evaluate the efficacy of treatment. Morphometric computerized semi-automated assessment of the quantitative degree of liver fibrosis based upon specific staining of collagen and/or serum markers of liver fibrosis can also be measured as an indication of the efficacy of a subject treatment method. Secondary indices of liver function include, but are not limited to, serum transaminase levels, prothrombin time, bilirubin, platelet count, portal pressure, albumin level, and assessment of the Child-Pugh score. An effective amount of antiviral agent is an amount that is effective to increase an index of liver function by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the index of liver function in an untreated individual, or to a placebo-treated individual. Those skilled in the art can readily measure such indices of liver function, using standard assay methods, many of which are commercially available, and are used routinely in clinical settings.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

A therapeutically effective amount of antiviral agent is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Those skilled in the art can readily measure such serum markers of liver fibrosis, using standard assay methods, many of which are commercially available, and are used routinely in clinical settings. Methods of measuring serum markers include immunological-based methods, e.g., enzyme-linked immunosorbent assays (ELISA), radioimmunoassays, and the like, using antibody specific for a given serum marker.

Quantitative tests of functional liver reserve can also be used to assess the efficacy of treatment with antiviral agent. These include: indocyanine green clearance (ICG), galactose elimination capacity (GEC), aminopyrine breath test (ABT), antipyrine clearance, monoethylglycine-xylidide (MEG-X) clearance, and caffeine clearance.

As used herein, a “complication associated with cirrhosis of the liver” refers to a disorder that is a sequellae of decompensated liver disease, i.e., or occurs subsequently to and as a result of development of liver fibrosis, and includes, but it not limited to, development of ascites, variceal bleeding, portal hypertension, jaundice, progressive liver insufficiency, encephalopathy, hepatocellular carcinoma, liver failure requiring liver transplantation, and liver-related mortality.

A therapeutically effective amount of antiviral agent is an amount that is effective in reducing the incidence (e.g., the likelihood that an individual will develop) of a disorder associated with cirrhosis of the liver by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to an untreated individual, or to a placebo-treated individual.

Whether treatment with antiviral agent is effective in reducing the incidence of a disorder associated with cirrhosis of the liver can readily be determined by those skilled in the art.

Reduction in liver fibrosis increases liver function. Thus, the invention provides methods for increasing liver function, generally involving administering a therapeutically effective amount of antiviral agent. Liver functions include, but are not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

Whether a liver function is increased is readily ascertainable by those skilled in the art, using well-established tests of liver function. Thus, synthesis of markers of liver function such as albumin, alkaline phosphatase, alanine transaminase, aspartate transaminase, bilirubin, and the like, can be assessed by measuring the level of these markers in the serum, using standard immunological and enzymatic assays. Splanchnic circulation and portal hemodynamics can be measured by portal wedge pressure and/or resistance using standard methods. Metabolic functions can be measured by measuring the level of ammonia in the serum.

Whether serum proteins normally secreted by the liver are in the normal range can be determined by measuring the levels of such proteins, using standard immunological and enzymatic assays. Those skilled in the art know the normal ranges for such serum proteins. The following are non-limiting examples. The normal range of alanine transaminase is from about 7 to about 56 units per liter of serum. The normal range of aspartate transaminase is from about 5 to about 40 units per liter of serum. Bilirubin is measured using standard assays. Normal bilirubin levels are usually less than about 1.2 mg/dL. Serum albumin levels are measured using standard assays. Normal levels of serum albumin are in the range of from about 35 to about 55 g/L. Prolongation of prothrombin time is measured using standard assays. Normal prothrombin time is less than about 4 seconds longer than control.

A therapeutically effective amount of antiviral agent is one that is effective to increase liver function by at least about 10%, at least about 20%, at least about 30%, at least about 40%, at least about 50%, at least about 60%, at least about 70%, at least about 80%, or more. For example, a therapeutically effective amount of antiviral agent is an amount effective to reduce an elevated level of a serum marker of liver function by at least about 10%, at least about 20%, at least about 30%, at least about 40%, at least about 50%, at least about 60%, at least about 70%, at least about 80%, or more, or to reduce the level of the serum marker of liver function to within a normal range. A therapeutically effective amount of IFN-γ is also an amount effective to increase a reduced level of a serum marker of liver function by at least about 10%, at least about 20%, at least about 30%, at least about 40%, at least about 50%, at least about 60%, at least about 70%, at least about 80%, or more, or to increase the level of the serum marker of liver function to within a normal range.

Method of Reducing Risk of Hepatic Cancer

The present invention provides methods of reducing the risk that an individual will develop hepatic cancer. The methods involve administering an antiviral agent, as describe above, wherein viral load is reduced in the individual, and wherein the risk that the individual will develop hepatic cancer is reduced. An effective amount of antiviral agent is one that reduces the risk of hepatic cancer by at least about 10%, at least about 20%, at least about 25%, at least about 30%, at least about 35%, at least about 40%, at least about 45%, at least about 50%, at least about 55%, at least about 60%, at least about 65%, at least about 70%, or more. Whether the risk of hepatic cancer is reduced can be determined in, e.g., study groups, where individuals treated according to the methods of the invention have reduced incidence of hepatic cancer.

Subjects Suitable for Treatment

Individuals who have been clinically diagnosed as infected with a hepatitis virus (e.g., HAV, HBV, HCV, delta, etc.), particularly HCV, are suitable for treatment with the methods of the instant invention. Individuals who are infected with HCV are identified as having HCV RNA in their blood, and/or having anti-HCV antibody in their serum. Such individuals include naïve individuals (e.g., individuals not previously treated for HCV, particularly those who have not previously received IFN-α-based or ribavirin-based therapy) and individuals who have failed prior treatment for HCV (“treatment failure” patients). Treatment failure patients include non-responders (e.g., individuals in whom the HCV titer was not significantly or sufficiently reduced by a previous treatment for HCV, particularly a previous IFN-α monotherapy using a single form of IFN-α); and relapsers (e.g., individuals who were previously treated for HCV (particularly a previous IFN-α monotherapy using a single form of IFN-α), whose HCV titer decreased significantly, and subsequently increased). In particular embodiments of interest, individuals have an HCV titer of at least about 10⁵, at least about 5×10⁵, or at least about 10⁶, genome copies of HCV per milliliter of serum. The patient may be infected with any HCV genotype (genotype 1, including 1a and 1b, 2, 3, 4, 6, etc. and subtypes (e.g., 2a, 2b, 3a, etc.)), particularly a difficult to treat genotype such as HCV genotype 1 and particular HCV subtypes and quasispecies.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### C-Terminal Pegylation of Consensus Interferon Alfacon-1

CIFN Alfacon-1 is dissolved at a concentration of 1-10 mg/ml in a suitable buffer system, either 100 mM morpholinoethane sulfonate (MES) at pH 4.7-6.0 or 10 mM sodium phosphate containing 100 mM sodium chloride at a pH 6.0-7.4. The coupling molecule, namely the PEG-NH₂ (linear or branched; MW 20-40 kDa) is dissolved in the same buffer as the CIFN and added to the protein solution such that the concentration of the PEG-NH₂ remains ten times the molar concentration of the protein. This ratio can be changed following an analysis of the product (mono PEGylation vs multiple PEGylation). A high stock concentration of the carbodiimide reagent (EDAC) in the same buffer or in water is prepared to a final concentration of 0.5-1.0 M. Sufficient quantities of this stock solution are added to the reaction vessel such that the ratio of the PEG reagent to EDAC is 1:1 on a molar basis. The reaction mixture is stirred with a magnetic stirrer and the reaction is allowed to proceed for 1-6 hours at ambient temperature (e.g., about 17° C.). The reaction may be monitored by a size-exclusion HPLC and the molar stoichiometry and the reaction temperature may be adjusted to optimize the formation of monopegylated derivative.

The reaction mixture is then purified by gel filtration or diafiltration to remove the excess reagent and products derived from them. If some turbidity is seen in the reaction mixture, the product(s) is additionally filtered prior to purification and analyzed for the contents. The filtrate is subjected to purification initially by diafiltration or gel filtration. Rigorous separations are achieved using HPLC methods. The product is finally characterized by mass spectrometry, protein sequence, peptide mapping and other techniques. The biological activity of the material is ascertained by a cytopathic protective effect inhibition assay.

The purified monopegylated product is formulated to contain 40-400 μg/ml of PEGylated interferon alpha in 10 mM sodium phosphate buffer containing 100 mM sodium chloride and 0.01-0.1% (w/v) polysorbate 20 or 80.

Adults (˜70 kg body weight) with chronic hepatitis C infection as indicated by detectable HCV RNA levels and elevated serum alanine aminotransferase levels and liver histopathology consistent with the disease are administered with 32.5 μg, 65 μg, 97.5 μg and 130 μg of the PEGylated interferon formulation supplied in aqueous buffer (injection volume of 0.5-1.0 ml) subcutaneously once a week for 48 weeks. The serum samples are withdrawn from the patients once a month and analyzed for the treatment efficacy based on RNA PCR determination, ALT level in serum. The assay monitoring is continued for 24 weeks past the cessation of therapy. Sustained viral response at 72 weeks is assessed on the basis of undetectable HCV RNA levels in blood and normalization of serum ALT levels.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method for treating hepatitis C virus infection in an individual, the method comprising:
administering a composition comprising a first form of interferon-α (IFN-α) and a second form of IFN-α, wherein said second form of IFN-α comprises a polyethylene glycol (PEG) moiety and, as a result, has a mean residence time that is greater than the mean residence time of the first form of IFN-α, which composition is administered in an amount effective to achieve a first serum concentration of IFN-α that is at least about 80% of the maximum tolerated dose (MTD) in International Units of IFN-α per milliliter of serum (IU/ml) within a first period of time of about 24 to 48 hours, followed by a second concentration of IFN-α that is about 50% or less than the MTD, which second concentration is maintained for a second period of time of at least seven days.

2. The method of claim 1, wherein a sustained viral response is achieved.

3. The method of claim 1, further comprising administering IFN-γ for a period of from about 1 day to about 14 days before administration of IFN-α.

4. The method of claim 1, wherein the second form of IFN-α comprises a PEG moiety covalently linked, directly or via a linker, to one or more amino acid side chains of amino acid residues 1-10 of the IFN-α polypeptide.

5. The method of claim 1, wherein the second form of IFN-α comprises a PEG moiety covalently linked, directly or via a linker, to the amino-terminal amino acid of the IFN-α polypeptide.

6. The method of claim 1, wherein the second form of IFN-α comprises a PEG moiety covalently linked, directly or via a linker, to one or more amino acid side chains of amino acid residues 150-166 of the IFN-α polypeptide.

7. The method of claim 1, wherein the second form of IFN-α comprises a PEG moiety covalently linked, directly or via a linker, to the carboxyl-terminal amino acid of the IFN-α polypeptide.

8. A method of treating hepatitis C virus infection in an individual, the method comprising:
administering a composition comprising a first form of interferon-α (IFN-α) and a second form of IFN-α, wherein said second form of IFN-α comprises a polyethylene glycol (PEG) moiety and, as a result, has a mean residence time that is greater than the mean residence time of the first form of IFN-α, wherein the composition is administered in an amount effective to achieve a first phase and a second phase, wherein, in the first phase, a first serum concentration of IFN-α is achieved that is at least about 80% of the maximum tolerated dose (MTD) in International Units of IFN-α per milliliter of serum (IU/ml) within a first period of time of about 24 hours, wherein in the second phase, the ratio of the highest IFN-α serum concentration to the lowest serum IFN-α concentration, measured over any 24-hour period during the second phase, is less than 3, and wherein the highest concentration of IFN-α during the second phase is about 50% or less than the MTD.

9. The method of claim 8, wherein the ratio of the highest IFN-α serum concentration to the lowest serum IFN-α concentration, measured over any 24-hour period during the second phase is about 1.

10. A method for treating hepatitis C virus infection in an individual, the method comprising:
administering a composition comprising a first form of consensus interferon-α (CIFN) and a second form of CIFN, wherein said second form of CIFN comprises a polyethylene glycol (PEG) moiety and, as a result, has a mean residence time that is greater than the mean residence time of the first form of CIFN, wherein the composition is administered in an amount effective to achieve a first serum concentration of CIFN that is at least about 80% of the maximum tolerated dose (MTD) in International Units of IFN-α per milliliter of serum (IU/ml) within a first period of time of about 24 hours, followed by a second concentration of CIFN that is about 50% or less than the MTD, which second concentration is maintained for a second period of time of at least seven days.

11. A method of treating hepatitis C virus infection in an individual, the method comprising:
administering a composition comprising a first form of consensus interferon-α (CIFN) and a second form of CIFN, wherein said second form of CIFN comprises a polyethylene glycol (PEG) moiety and, as a result, has a mean residence time that is greater than the mean residence time of the first form of CIFN, wherein the composition is administered in an amount effective to achieve a first phase and a second phase, wherein, in the first phase, a first serum concentration of CIFN is achieved that is at least about 80% of the maximum tolerated dose (MTD) in International Units of IFN-α per milliliter of serum (IU/ml) within a first period of time of about 24 hours, wherein in the second phase, the ratio of the highest CIFN serum concentration to the lowest serum CIFN concentration, measured over any 24-hour period during the second phase, is less than 3, and wherein the highest concentration of CIFN during the second phase is about 50% or less than the MTD.

12. A method of treating hepatitis C virus infection in an individual, the method comprising:
administering IFN-α in a dosing regimen comprising a first phase and a second phase, wherein, in the first phase, a first serum concentration C1ₘₐₓ of IFN-α in International Units of IFN-α per milliliter of serum (IU/ml) is achieved within a first period of time of about 24 hours, wherein in the second phase, a second serum concentration Csus in International Units of IFN-α per milliliter of serum (IU/ml) is achieved that is about 50% of C1max or less, and wherein the area under the curve, defined by IFN-α serum concentration as a function of time, during any 24-hour time period in the second phase is no greater than the area under the curve of day 2 to day 3 as shown in FIG. 2.

13. A method of treating hepatitis C virus infection in an individual, the method comprising:
administering consensus IFN-α (CIFN) in a dosing regimen comprising a first phase and a second phase, wherein, in the first phase, a first serum concentration C1 max of CIFN in International Units of IFN-α per milliliter of serum (IU/ml) is achieved within a first period of time of about 24 hours, wherein in the second phase, a second concentration Csus of CIFN in International Units of IFN-α per milliliter of serum (IU/ml) is achieved that is about 50% of C1max or less, and wherein the area under the curve, defined by CIFN serum concentration as a function of time, during any 24-hour time period in the second phase is no greater than the area under the curve of day 2 to day 3 as shown in FIG. 2.

14. A composition comprising:
a first form of interferon-α (IFN-α), wherein the covalent molecular structure of the first form of IFN-α comprises a first IFN-α polypeptide free of polyethylene glycol;
a second form of IFN-α, wherein the covalent molecular structure of the second form of IFN-α comprises a second IFN-α polypeptide covalently linked, directly or via a linker, to a polyethylene glycol (PEG) moiety; and
a pharmaceutically acceptable excipient.

15. The composition of claim 14, wherein in the second form of IFN-α, the PEG moiety is covalently linked, directly or via a linker, to one or more amino acid side chains of amino acid residues 1-10 of the second IFN-α polypeptide.

16. The composition of claim 14, wherein in the second form of IFN-α, the PEG moiety is covalently linked, directly or via a linker, to the amino-terminal amino acid of the second IFN-α polypeptide.

17. The composition of claim 16, wherein the PEG moiety is covalently linked, directly or via a linker, to the α-amino group of the amino-terminal amino acid of the second polypeptide.

18. The composition of claim 17, wherein the PEG moiety is covalently linked, directly or via a linker, by an amide bond to the α-amino group of the amino-terminal amino acid of the second polypeptide.

19. The composition of claim 14, wherein in the second form of IFN-α, the PEG moiety is covalently linked, directly or via a linker, to one or more amino acid side chains of amino acid residues 150-166 of the second IFN-α polypeptide.

20. The composition of claim 14, wherein in the second form of IFN-α, the PEG moiety is covalently linked, directly or via a linker, to the carboxyl-terminal amino acid of the second IFN-α polypeptide.

21. The composition of claim 20, wherein the PEG moiety is covalently linked, directly or via a linker, to the α-carboxyl group of the carboxyl-terminal amino acid of the second IFN-α polypeptide.

22. The composition of claim 21, wherein the PEG moiety is covalently linked, directly or via a linker, by an amide bond to the α-carboxyl group of the carboxyl-terminal amino acid of the second IFN-α polypeptide.

23. The composition of any of claims 19-22, wherein the covalent molecular structure of the second form of IFN-α comprises no PEG moiety that is linked, directly or via a linker, to an amino acid in amino acid residues 1-149 of the second IFN-α polypeptide.

24. The composition of any of claims 15-18, wherein the second form of IFN-α comprises a single PEG moiety.

25. The composition of any of claims 19-22, wherein the second form of IFN-α comprises a single PEG moiety.

26. The composition of claim 23, wherein the second form of IFN-α comprises a single PEG moiety.

27. The composition of claim 26, wherein the first form of IFN-α and the second form of IFN-α are present at a molar ratio of about 1:1 in the composition.

28. The composition of claim 25, wherein the first form of IFN-α and the second form of IFN-α each comprise a single IFN-α polypeptide molecule, wherein the IFN-α polypeptide is the same for the first and second forms and is selected from the group consisting of IFN-α-2a, IFN-α-2b and consensus IFN-α polypeptides.

29. The composition of claim 28, wherein the IFN-α polypeptide is selected from the group consisting of consensus IFN-α polypeptides.

30. The composition of claim 27, wherein the first form of IFN-α and the second form of IFN-α each comprise a single IFN-α polypeptide molecule, wherein the IFN-α polypeptide is the same for the first and second forms and is selected from the group consisting of consensus IFN-α polypeptides.

31. The composition of any of claims 15-18, wherein the first form of IFN-α and the second form of IFN-α are present at a molar ratio of about 1:5 in the composition.

32. The composition of any of claims 15-18 wherein the first form of IFN-α and the second form of IFN-α each comprise a single IFN-α polypeptide molecule, wherein the IFN-α polypeptide is the same for the first and second forms and is selected from the group consisting of IFN-α-2a, IFN-α-2b and consensus IFN-α polypeptides.

33. The composition of claim 32, wherein the IFN-α polypeptide is selected from the group consisting of consensus IFN-α polypeptides.

34. The composition of claim 33, wherein the second form of IFN-α comprises a single PEG moiety.

35. The composition of claim 31, wherein the first form of IFN-α and the second form of IFN-α each comprise a single IFN-α polypeptide molecule, wherein the IFN-α polypeptide is the same for the first and second forms and is selected from the group consisting of consensus IFN-α polypeptides, and wherein the second form of IFN-α comprises a single PEG moiety.

36. An interferon-α (IFN-α) derivative comprising a single interferon-α (IFN-α) polypeptide, wherein the IFN-α polypeptide is covalently linked, directly or via a linker, to one or more polyethylene glycol (PEG) moieties, wherein the IFN-α polypeptide is linked to each PEG moiety at one or more sites at or near the carboxyl-terminus of the IFN-α polypeptide, and wherein the IFN-α polypeptide is covalently linked, directly or via a linker, to no PEG moiety at any site other than a site at or near the carboxyl-terminus of the IFN-α polypeptide.

37. The interferon-α (IFN-α) derivative of claim 36, wherein the IFN-α polypeptide is covalently linked, directly or via a linker, to no amino acid in amino acid residues 1-149 of the IFN-α polypeptide.

38. The interferon-α (IFN-α) derivative of claim 36 or 37, wherein at least one PEG moiety is covalently linked, directly or via a linker, to one or more amino acid side chains of amino acid residues 150-166 of the IFN-α polypeptide.

39. The interferon-α (IFN-α) derivative of claim 36 or 37, wherein at least one PEG moiety is covalently linked, directly or via a linker, to the carboxyl-terminal amino acid of the IFN-α polypeptide.

40. The interferon-α (IFN-α) derivative of claim 39, wherein at least one PEG moiety is covalently linked, directly or via a linker, to the α-carboxyl group of the carboxyl-terminal amino acid of the IFN-α polypeptide.

41. The interferon-α (IFN-α) derivative of claim 40, wherein at least one PEG moiety is covalently linked, directly or via a linker, by an amide bond to the α-carboxyl group of the carboxyl-terminal amino acid of the IFN-α polypeptide.

42. An interferon-α (IFN-α) derivative comprising a single interferon-α (IFN-α) polypeptide, wherein the IFN-α polypeptide is either (1) covalently linked to a single PEG moiety directly, or via a linker, via a single covalent bond located at or near the carboxyl-terminus of the IFN-α polypeptide or (2) covalently linked to a plurality of PEG moieties via a linker and via a single covalent bond between the linker and the IFN-α polypeptide, wherein the bond is located at a site at or near the carboxyl-terminus of the IFN-α polypeptide.

43. The interferon-α (IFN-α) derivative of claim 42, wherein each PEG moiety is covalently linked, directly or via a linker, to an amino acid side chain of an amino acid in amino acid residues 150-166 of the IFN-α polypeptide.

44. The interferon-α (IFN-α) derivative of claim 42, wherein each PEG moiety is covalently linked, directly or via a linker, to the carboxyl-terminal amino acid of the IFN-α polypeptide.

45. The interferon-α (IFN-α) derivative of claim 44, wherein each PEG moiety is covalently linked, directly or via a linker, to the α-carboxyl group of the carboxyl-terminal amino acid of the IFN-α polypeptide.

46. The interferon-α (IFN-α) derivative of claim 45, wherein each PEG moiety is covalently linked, directly or via a linker, by an amide bond to the α-carboxyl group of the carboxyl-terminal amino acid of the IFN-α polypeptide.

47. The interferon-α (IFN-α) derivative of any of claims 42-46, wherein the IFN-α polypeptide is covalently linked to a single PEG moiety.

48. The interferon-α (IFN-α) derivative of claim 47, wherein the IFN-α polypeptide is a consensus interferon-α polypeptide.

49. The interferon-α (IFN-α) derivative of claim 47, wherein the IFN-α polypeptide is an interferon-α-2a polypeptide.

50. The interferon-α (IFN-α) derivative of claim 47, wherein the IFN-α polypeptide is an interferon-α-2b polypeptide.

51. A composition comprising the interferon-α (IFN-α) derivative of claim 47 and a pharmaceutically acceptable excipient.

52. The composition of claim 51, wherein the IFN-α polypeptide is a consensus interferon-α polypeptide.

53. A method of treating hepatitis C infection in an individual, comprising administering an effective amount of the composition of claim 51 to the individual.

54. A method of treating hepatitis C infection in an individual, comprising administering an effective amount of the composition of claim 52 to the individual.

55. The method of claim 54, wherein the administration of the composition of claim 52 delivers to the individual a total of about 5,000,000 to 10,000,000 International Units of interferon-α.

56. A method of treating hepatitis C infection in an individual, comprising administering an effective amount of the composition of claim 14 to the individual.

57. A method of treating hepatitis C infection in an individual, comprising administering an effective amount of the composition of claim 30 to the individual.

58. The method of claim 57, wherein the administration of the composition of claim 30 delivers to the individual a total of about 5,000,000 to 10,000,000 International Units of interferon-α.

59. A method of treating hepatitis C infection in an individual, comprising administering an effective amount of the composition of claim 35 to the individual.

60. The method of claim 59, wherein the administration of the composition of claim 30 delivers to the individual a total of about 5,000,000 to 10,000,000 International Units of interferon-α.
